(19) United States
(12) Patent Application Publication
(10) Pub. No.: US 2026/0085280 A1
(43) Pub. Date: Mar. 26, 2026

(54) ADAPTATION AND PROCESS OPTIMIZATION OF MICROORGANISMS FOR GROWTH IN HEMICELLULOSIC DERIVED CARBOHYDRATES

(71) Applicant: MARA RENEWABLES CORPORATION, Dartmouth (CA)

(72) Inventors: Kimberly Hyson, Dartmouth (CA); Nathalia Florez, Dartmouth (CA); Denise Muise, Dartmouth (CA); Alexandra Merkx-Jacques, Dartmouth (CA); Dorothy Dennis, Dartmouth (CA); David Woodhall, Dartmouth (CA); Zachary Sun, Dartmouth (CA); Roberto E. Armenta, Dartmouth (CA)

(73) Assignee: MARA RENEWABLES CORPORATION, Dartmouth (CA)

(21) Appl. No.: 19/350,363

(22) Filed: Oct. 6, 2025

Related U.S. Application Data
(63) Continuation of application No. 17/286,449.
(60) Provisional application No. 62/749,554, filed on Oct. 23, 2018.

(51) Int. Cl. C12N 1/22; C12N 1/36; C12N 9/12; C12N 9/90

(52) U.S. Cl. CPC C12N 1/22; C12N 1/36; C12N 9/1205; C12N 9/90; C12Y 207/01017; C12Y 503/01005

(57) ABSTRACT

Provided herein are methods of making microorganisms modified for increased xylose consumption as compared to unmodified microorganisms. The methods include providing xylose-consuming microorganisms comprising two or more copies of a nucleic acid sequence encoding xylose isomerase and two or more copies of a nucleic acid sequence encoding a xylose kinase, culturing the microorganisms in medium containing xylose and harvesting a portion of the microorganisms. These steps are repeated multiple times. The microorganisms are then isolated. The isolated microorganisms have increased xylose consumption rates compared to control xylose-consuming microorganisms. Also provided are a population of microorganisms made by the provided methods. Methods of culturing the population of microorganisms and methods of reducing xylitol production in cultures comprising the population of microorganisms are provided.

### CROSS-REFERENCE TO RELATED APPLICATIONS

This application is a continuation of U.S. Non-Provisional application Ser. No. 17/286,449, filed Apr. 16, 2021, which is a national stage of PCT Application No. PCT/IB2019/058953, filed Oct. 21, 2019 which claims priority to U.S. Provisional Application No. 62/749,554, filed Oct. 23, 2018, which are incorporated by reference herein in their entirety.

### SEQUENCE LISTING

The instant application contains a Sequence Listing in XML format submitted electronically and is hereby incorporated by reference in its entirety. The Sequence Listing file is entitled “095523-1482723-021US2.xml” is 31,135 bytes in size and was created on Oct. 6, 2025.

### BACKGROUND

Eukaryotic microorganisms can be used to produce lipids by converting carbon provided in the culture medium to lipids. These lipids can then be harvested from the microorganisms and used in a variety of ways, including for production of nutritional oils and biofuel. Typically, the carbon provided in the culture medium is glucose. However, glucose is an expensive medium component. Cheaper carbon sources can be obtained from lignocellulose materials by converting cellulosic and hemicellulosic components into two main streams hemicellulosic glucose and hemicellulosic xylose. However, xylose, in most cases, cannot be metabolized and, thus, is often regarded as waste.

### BRIEF SUMMARY

Provided herein are methods of making microorganisms modified for increased xylose consumption as compared to unmodified microorganisms. The methods include providing xylose-consuming microorganisms comprising two or more copies of a nucleic acid sequence encoding xylose isomerase and two or more copies of a nucleic acid sequence encoding a xylose kinase, culturing the microorganisms in medium containing xylose and harvesting a portion of the microorganisms. These steps are repeated multiple times. The microorganisms are then isolated. The isolated microorganisms have increased xylose consumption rates compared to control xylose-consuming microorganisms. Also provided are a population of microorganisms made by the provided methods. Methods of culturing the population of microorganisms and methods of reducing xylitol production in cultures comprising the population of microorganisms are provided.

### BRIEF DESCRIPTION OF THE DRAWINGS

FIGS. 1A, 1B, 1C, 1D, 1E, and IF are graphs showing xylose depletion of Iso-his #16, 7-7, Gxs1 7-7, AspTx 7-7 and 51-7 in various media showing improvement over wild type (unmodified) microorganisms. See Table 1 for strain description. FIGS. 1A and 1B are graphs showing xylose consumption and the amount of conversion of xylose to xylitol, respectively, when microorganisms were grown on laboratory-grade 20 g/L glucose and 20 g/L xylose (2:2 GX). FIGS. 1C and 1D are graphs showing xylose consumption and the amount of conversion of xylose to xylitol, respectively, when microorganisms were grown on laboratory-grade 20 g/L glucose and 50 g/L xylose (2:5 GX). FIGS. 1E and 1F show xylose consumption and the amount of conversion of xylose to xylitol, respectively, when microorganisms were grown on laboratory-grade 60 g/L xylose (6% xylose).

FIGS. 2A, 2B, 2C, and 2D are graphs showing the impact of xylitol addition on glucose and xylose use by 7-7 and 51-7 strains grown in laboratory-grade carbon sources at concentrations of 2% glucose, 5% xylose or 2:5 glucose:xylose. FIG. 2A is a graph showing glucose consumed by 7-7 and 51-7 grown in 2% glucose (2% G), 2% glucose with 1 g/L xylitol or 2% glucose with 15 g/L xylitol. FIG. 2B is a graph showing xylose consumed by 7-7 and 51-7 grown in 5% xylose, 5% xylose with 1 g/L xylitol or 5% xylose with 15 g/L xylitol. FIGS. 2C and 2D are graphs showing glucose used (2C) and xylose used (2D) by 7-7 and 51-7 grown in 2:5 glucose:xylose (2:5 GX), 2:5 GX with 1 g/L xylitol or 2:5 GX with 15 g/L xylitol.

FIGS. 3A, 3B and 3C are graphs showing fermentations with Gxs1 7-7 and 51-7 in medium containing hemicellulosic xylose. FIG. 3A is a graph showing biomass accumulation of 7-7 and Gxs1 7-7 grown in medium containing hemicellulosic xylose.

FIGS. 3B and 3C are graphs showing carbon consumption and xylitol accumulation by 51-7 (3B) and Gxs1 7-7 (3C) strains grown in medium containing hemicellulosic xylose.

FIG. 4 is a graph showing nitrogen concentration affects xylitol production in wild type unmodified ONC-T18, 7-7 and 51-7 strains.

FIGS. 5A and 5B are graphs showing passaging of 7-7 and AspTx 7-7 strains resulted in strains with increased xylose usage in both 5% xylose (5A) and 2:5 glucose:xylose (5B). Carbon sources were laboratory-grade.

FIGS. 6A, 6B, 6C, and 6D are graphs showing xylose use and xylitol production and biomass production in passaged 51-7 strains grown in laboratory-grade carbon sources at concentrations of 5% xylose (5% Xyl) and 2:5 glucose:xylose (2:5% Glc:Xyl). FIG. 6A is a graph showing xylose used when passaged strains were grown on 5% xylose. FIG. 6B is a graph showing xylose used when passaged strains were grown on 2:5 glucose:xylose. FIG. 6C is a graph showing xylitol production by passaged strains. FIG. 6D is a graph showing biomass production of passaged strains grown on 5% xylose or 2:5 glucose:xylose.

FIGS. 7A and 7B are graphs showing xylose used (7A) and xylitol produced (7B) by 51-7 original and 51-7 passaged strains.

FIGS. 8A, 8B and 8C are an image and graphs showing relative xylose isomerase and pirXK copy numbers in 51-7 passaged strains. FIG. 8A are images of Southern blots showing xylose isomerase and pirXK genes and IMP loading control. FIG. 8B is a graph of the relative xylose isomerase intensities from the Southern blot. FIG. 8C is a graph of the relative pirXK intensities of the Southern blot.

FIGS. 9A, 9B, and 9C are graphs showing the effect of increasing hemicellulosic xylose concentrations on cultures of 51-7 and 51-7 XP16 (strain isolated after 16 passages). FIG. 9A is a graph showing the amount of xylose used when strains were cultured in 20, 30, 40, or 50 g/L hemicellulosic xylose. FIG. 9B is a graph showing the amount of glucose used when strains were cultured in various amounts of hemicellulosic xylose. FIG. 9C is a graph showing the amount of xylitol produced when strains were cultured in various amounts of hemicellulosic xylose.

FIGS. 10A and 10B are graphs showing the effect of increasing hemicellulosic glucose concentrations on 51-7 and 51-7 XP16 cultures. FIG. 10A is a graph showing the amount of glucose used when strains were cultured in 30, 40, or 50 g/L hemicellulosic glucose. FIG. 10B is a graph showing the amount of xylose used when strains were cultured in 30, 40, or 50 g/L hemicellulosic glucose.

FIGS. 11A, 11B and 11C are graphs showing benchmark fermentations using 51-7 XP16 strain. FIG. 11A is a graph showing biomass growth of 51-7 XP16 grown in duplicate vessels (vessel A and vessel B) with laboratory-grade xylose and glucose as feedstock. FIG. 11B is a graph showing the amount of carbon consumption in vessel A. FIG. 11C is a graph showing the amount of carbon consumption in vessel B.

FIGS. 12A and 12B are tables showing the fatty acid profile of 51-7 XP16 in vessel A (12A) and vessel B (12B) grown on laboratory-grade carbohydrates.

FIG. 13 is a graph showing biomass growth of 51-7 XP16 with double the nitrogen concentration and hemicellulosic xylose and hemicellulosic glucose (51-7 XP16 C5/C6) or with double nitrogen and hemicellulosic xylose (51-7 XP16 C5).

FIGS. 14A and 14B are tables showing the fatty acid profiles of 51-7 XP16 grown in hemicellulosic xylose and hemicellulosic glucose (14A) and in only hemicellulosic xylose (14B).

FIG. 15 is a table showing the biomass growth and fatty acid profile of 51-7 XP16 at 3200 L scale grown on hemicellulosic glucose.

FIG. 16 is a graph showing glucose and xylose consumption and dissolved oxygen profile of 51-7 XP16 at 3200 L scale grown on hemicellulosic glucose.

FIG. 17 is a graph showing the amount of xylose used by 51-7 XP16 compared to wild type strain ONC-T18 at 3200 L grown on hemicellulosic glucose.

### DETAILED DESCRIPTION

In nature, two xylose metabolism pathways exist, the xylose reductase/xylitol dehydrogenase pathway and the xylose isomerase/xylulose kinase pathway. Thraustochytrids have genes that encode proteins active in both pathways; however, the former pathway appears to be dominant as evidenced by a build-up of xylitol when grown in a xylose medium. Thus, strains were generated that over-express xylose isomerases, xylulose kinases and/or xylose transporters as described in U.S. Publication No. 2017/0015988, which is incorporated by reference herein in its entirety. As described herein these strains were further optimized using laboratory adaptation in medium containing xylose either as the sole carbon source or in medium containing xylose and glucose. A representative passaged strain, 51-7 XP16, used 2.4-fold more xylose than the unpassaged, original strain (51-7 original) in media containing both laboratory-grade glucose and xylose and 5.5-fold more xylose than 51-7 in media containing laboratory-grade xylose only (See Table 1 for strain description). As used herein laboratory grade carbon sources are carbon sources containing 95% or greater of the carbon source, e.g., a laboratory-grade glucose contains 95% or greater glucose. 51-7 XP16 also produced approximately 8-fold less xylitol than the original strain in both media. In medium containing hemicellulosic xylose, 51-7 XP16 used 1.2- to 8.8-fold more xylose than the 51-7 original strain depending on the amount of hemicellulosic xylose provided. Further, 51-7 XP16's ability to use glucose in media containing hemicellulosic glucose was not hindered.

Provided herein is a method of making microorganisms with increased xylose consumption. The method includes (a) providing xylose-consuming microorganisms comprising two or more copies of a nucleic acid sequence encoding xylose isomerase and two or more copies of a nucleic acid sequence encoding a xylose kinase; (b) culturing the microorganisms in a first culture medium comprising xylose for at least 3 days; (c) harvesting a portion of the microorganisms from the first culture medium after culture step (b); (d) culturing the harvested portion of microorganisms in a second culture medium comprising xylose for at least 3 days; (c) harvesting a portion of the microorganisms from the second culture medium after culture step (d); (f) repeating culturing and harvesting steps (d) and (c) at least two times in a third culture medium and a fourth culture medium; and (g) isolating the harvested microorganisms from step (f), wherein the isolated microorganisms have increased xylose consumption rates compared to control xylose-consuming microorganisms.

As described herein, a control or standard control refers to a sample, measurement, or value that serves as a reference, usually a known reference, for comparison to a test sample, measurement, or value. For example, a test microorganism, e.g., a microorganism made by the provided methods with increased xylose consumption and encoding genes for metabolizing xylose can be compared to a known normal (wild-type) microorganism (e.g., a standard control microorganism) or an unpassaged, original strain that has not been subjected to the provided methods, e.g., a control-xylose consuming microorganism. A standard control can also represent an average measurement or value gathered from a population of microorganisms (e.g., standard control microorganisms) that do not grow or grow poorly on xylose as the sole carbon source or that do not have or have minimal levels of xylose isomerase activity, xylulose kinase activity and/or xylose transport activity. One of skill will recognize that standard controls can be designed for assessment of any number of parameters (e.g., RNA levels, polypeptide levels, specific cell types, and the like).

The provided strains have nucleic acids encoding one or more genes involved in xylose metabolism. Thus, provided herein are nucleic acids and polypeptides encoding xylose isomerase, xylulose kinase and xylose transporters for modifying microorganisms to be capable of metabolizing xylose and/or growing on xylose as the sole carbon source. Thus, provided are nucleic acids encoding a xylose isomerase. The nucleic acid sequences can be endogenous or heterologous to the microorganism. Exemplary nucleic acids sequences of xylose isomerases include, but are not limited to, those from Piromyces sp., Streptococcus sp., and Thraustochytrids. For example, exemplary nucleic acid sequences encoding xylose isomerases include, but are not limited to, SEQ ID NO:2 and SEQ ID NO:4; and exemplary polypeptide sequences of xylose isomerase include, but are not limited to, SEQ ID NO:5. Exemplary nucleic acid sequences of xylulose kinases include, but are not limited to, those from E. coli, Piromyces sp., Saccharomyces sp., and Pichia sp. For example, exemplary nucleic acid sequences encoding xylulose kinases include, but are not limited to, SEQ ID NO: 3, SEQ ID NO:6, SEQ ID NO:7, and SEQ ID NO:8. Exemplary nucleic acid sequences encoding sugar transporters, e.g., xylose transporters, include, but are not limited to, those from Aspergillus sp., Gfx1, Gxs1 and Sut1. For example, exemplary nucleic acid sequences encoding xylose transporters include, but are not limited to, SEQ ID NO:9, SEQ ID NO:10, SEQ ID NO:11, and SEQ ID NO:12.

Optionally, the provided xylose-consuming microorganisms contain at least two copies of a nucleic acid sequence encoding a xylose isomerase and two or more copies of a nucleic acid sequence encoding a xylulose kinase. Optionally, the xylose-consuming microorganisms comprise at least one nucleic acid sequence encoding a xylose transporter. The nucleic acid sequences encoding the xylose isomerase, xylulose kinase, and/or xylose transporter are, optionally, exogenous nucleic acid sequences. Optionally, the nucleic acid sequence encoding the xylose isomerase is an endogenous nucleic acid sequence. Optionally, the nucleic acid sequence encoding the xylulose kinase and/or xylose transporter is a heterologous nucleic acid. Optionally, the microorganism contains at least two copies of a nucleic acid sequence encoding a xylose isomerase, at least two copies of a nucleic acid sequence encoding a xylulose kinase, and at least one nucleic acid sequence encoding a xylose transporter. Optionally, the heterologous nucleic acid sequence encoding the xylose isomerase is at least 90% identical to SEQ ID NO:2. Optionally, the heterologous nucleic acid sequence encoding the xylulose kinase is at least 90% identical to SEQ ID NO:5. As noted above, optionally, the nucleic acid encoding the xylose transporter is a heterologous nucleic acid. Optionally, the xylose transporter encoded by the heterologous nucleic acid is GXS1 from Candida intermedia. Optionally, the xylose transporter encoded by the heterologous nucleic acid is AspTX from Aspergillus sp. Optionally, the heterologous nucleic acid sequence encoding the xylose transporter is at least 90% identical to SEQ ID NO:11 or SEQ ID NO:9.

As used herein, the term heterologous refers to a nucleic acid sequence that is not native to a cell, i.e., is from a different organism than the cell. The terms exogenous and endogenous or heterologous are not mutually exclusive. Thus, a nucleic acid sequence can be exogenous and endogenous, meaning the nucleic acid sequence can be introduced into a cell but have a sequence that is the same as, or similar to, the sequence of a nucleic acid naturally present in the cell. Similarly, a nucleic acid sequence can be exogenous and heterologous meaning the nucleic acid sequence can be introduced into a cell but have a sequence that is not native to the cell, e.g., a sequence from a different organism. As used herein, the term endogenous, refers to a nucleic acid sequence that is native to a cell.

The provided recombinant microorganisms not only contain nucleic acid sequences encoding genes involved in xylose metabolism, they can include multiple copies of such sequences. Thus, the microorganism comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 copies of the nucleic acid sequence encoding xylose isomerase. Optionally, the microorganism comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 copies of the nucleic acid sequence encoding the xylulose kinase. Optionally, the microorganism comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 copies of the nucleic acid sequence encoding the xylose transporter. The multiple copies or subset thereof are optionally encoded within a single sequence. Additionally, the nucleic acid sequence optionally contains one or more linker residues or sequences between the multiple copies or subset thereof.

Nucleic acid, as used herein, refers to deoxyribonucleotides or ribonucleotides and polymers and complements thereof. The term includes deoxyribonucleotides or ribonucleotides in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, conservatively modified variants of nucleic acid sequences (e.g., degenerate codon substitutions) and complementary sequences can be used in place of a particular nucleic acid sequence recited herein. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

A nucleic acid is operably linked when it is placed into a functional relationship with another nucleic acid sequence. For example, DNA that encodes a presequence or secretory leader is operably linked to DNA that encodes a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the sequences being linked are near each other, and, in the case of a secretory leader, contiguous and in reading phase. However, enhancers do not have to be contiguous. For example, a nucleic acid sequence that is operably linked to a second nucleic acid sequence is covalently linked, either directly or indirectly, to such second sequence, although any effective three-dimensional association is acceptable. A single nucleic acid sequence can be operably linked to multiple other sequences. For example, a single promoter can direct transcription of multiple RNA species. Linking can be accomplished by ligation at convenient restriction sites. If such sites do not exist, the synthetic oligonucleotide adaptors or linkers are used in accordance with conventional practice.

The terms identical or percent identity, in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be substantially identical. This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A comparison window, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981); by the homology alignment algorithm of Needleman & Wunsch, J. Mol. Biol. 48:443 (1970); by the search for similarity method of Pearson & Lipman, Proc. Nat'l. Acad. Sci. USA 85:2444 (1988); by computerized implementations of these algorithms (GAP, BESTFIT, FASTA, and TFASTA in the Wisconsin Genetics Software Package, Genetics Computer Group, 575 Science Dr., Madison, WI); or by manual alignment and visual inspection (see, e.g., Current Protocols in Molecular Biology (Ausubel et al., eds. 1995 supplement)).

A preferred example of an algorithm that is suitable for determining percent sequence identity and sequence similarity are the BLAST and BLAST 2.0 algorithms, which are described in Altschul et al., Nuc. Acids Res. 25:3389-3402 (1977), and Altschul et al., J. Mol. Biol. 215:403-410 (1990), respectively. BLAST and BLAST 2.0 are used, with the parameters described herein, to determine percent sequence identity for nucleic acids or proteins. Software for performing BLAST analyses is publicly available through the National Center for Biotechnology Information, as known in the art. This algorithm involves first identifying high scoring sequence pairs (HSPs) by identifying short words of a selected length (W) in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The Expectation value (E) represents the number of different alignments with scores equivalent to or better than what is expected to occur in a database search by chance. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, expectation (E) of 10, and the BLOSUM62 scoring matrix (see Henikoff & Henikoff, Proc. Natl. Acad. Sci. USA 89:10915 (1989)), alignments (B) of 50, expectation (E) of 10, M=5, N=−4, and a comparison of both strands.

The term polypeptide, as used herein, generally has its art-recognized meaning of a polymer of at least three amino acids and is intended to include peptides and proteins. However, the term is also used to refer to specific functional classes of polypeptides, such as, for example, desaturases, elongases, etc. For each such class, the present disclosure provides several examples of known sequences of such polypeptides. Those of ordinary skill in the art will appreciate, however, that the term polypeptide is intended to be sufficiently general as to encompass not only polypeptides having the complete sequence recited herein (or in a reference or database specifically mentioned herein), but also to encompass polypeptides that represent functional fragments (i.e., fragments retaining at least one activity) of such complete polypeptides. Moreover, those in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term polypeptide as used herein. Those in the art can determine other regions of similarity and/or identity by analysis of the sequences of various polypeptides described herein. As is known by those in the art, a variety of strategies are known, and tools are available, for performing comparisons of amino acid or nucleotide sequences in order to assess degrees of identity and/or similarity. These strategies include, for example, manual alignment, computer assisted sequence alignment and combinations thereof. A number of algorithms (which are generally computer implemented) for performing sequence alignment are widely available, or can be produced by one of skill in the art. Representative algorithms include, e.g., the local homology algorithm of Smith and Waterman (Adv. Appl. Math., 1981, 2:482); the homology alignment algorithm of Needleman and Wunsch (J. Mol. Biol., 1970, 48:443); the search for similarity method of Pearson and Lipman (Proc. Natl. Acad. Sci. (USA), 1988, 85:2444); and/or by computerized implementations of these algorithms (e.g., GAP, BESTFIT, FASTA, and TFASTA in the Wisconsin Genetics Software Package Release 7.0, Genetics Computer Group, 575 Science Dr., Madison, Wis.). Readily available computer programs incorporating such algorithms include, for example, BLASTN, BLASTP, Gapped BLAST, PILEUP, CLUSTALW, etc. When utilizing BLAST and Gapped BLAST programs, default parameters of the respective programs may be used. Alternatively, the practitioner may use non-default parameters depending on his or her experimental and/or other requirements (see for example, the Web site having URL www.ncbi.nlm.nih.gov).

The provided xylose-consuming microorganisms with the nucleic acids encoding the genes involved in xylose metabolism and nucleic acid constructs containing the same include, but are not limited to, algae (e.g., microalgae), fungi (including yeast), bacteria, or protists. The microorganisms are optionally selected from the genus Oblongichytrium, Aurantiochytrium, Thraustochytrium, Schizochytrium, and Ulkenia or any mixture thereof. Optionally, the population of microorganisms includes Thraustochytriales as described in U.S. Pat. Nos. 5,340,594 and 5,340,742, which are incorporated herein by reference in their entireties. The microorganism can be a Thraustochytrium species, such as the Thraustochytrium species deposited as ATCC Accession No. PTA-6245 (i.e., ONC-T18) as described in U.S. Pat. No. 8,163,515, which is incorporated by reference herein in its entirety. Thus, the microorganism can have an 18s rRNA sequence that is at least 95%, 96%, 97%, 98%, 99%, 99.1%, 99.2%, 99.3%, 99.4%, 99.5%, 99.6%, 99.7%, 99.8%, 99.9% or more (e.g., including 100%) identical to SEQ ID NO:1. Optionally, the microorganisms are of the family Thraustochytriaceae. The microorganism can be a Thraustochytrium species, such as the Thraustochytrium species deposited as ATCC Accession No. PTA-6245 (i.e., ONC-T18), as described in U.S. Pat. No. 8,163,515, which is incorporated by reference herein in its entirety. The microorganisms can be ONC-T18.

The term thraustochytrid, as used herein, refers to any member of the order Thraustochytriales, which includes the family Thraustochytriaceae. Strains described as thraustochytrids include the following organisms: Order: Thraustochytriales; Family: Thraustochytriaceae; Genera: Thraustochytrium (Species: sp., arudimentale, aureum, benthicola, globosum, kinnei, motivum, multirudimentale, pachydermum, proliferum, roseum, striatum), Ulkenia (Species: sp., amoeboidea, kerguelensis, minuta, profunda, radiata, sailens, sarkariana, schizochytrops, visurgensis, yorkensis), Schizochytrium (Species: sp., aggregatum, limnaceum, mangrovei, minutum, octosporuni), Japoniochytrium (Species: sp., marinum), Aplanochytrium (Species: sp., haliotidis, kerguelensis, profunda, stocchinol), Althornia (Species: sp., crouchii), or Elina (Species: sp., marisalba, sinorifica). Species described within Ulkenia are considered to be members of the genus Thraustochytrium. Strains described as being within the genus Thraustochytrium may share traits in common with and also be described as falling within the genus Schizochytrium. For example, in some taxonomic classifications ONC-T18 may be considered within the genus Thraustochytrium, while in other classifications it may be described as within the genus Schizochytrium because it comprises traits indicative of both genera.

In the provided methods of making strains with increased xylose consumption, the microorganisms can be cultured for one or more days. Optionally, the microorganisms are cultured for 1, 2, 3, 4, 5, 6, 7, 8, 9, or 10 days in one or more of the culturing steps. Optionally, the microorganisms are cultured from 3 to 7 days in one or more culturing steps. The number of days the microorganisms are cultured in a particular culture step can be the same number of days or a different number of days from any other culturing step. For example, the microorganisms can be cultured for 3 days in the first culture medium and can be cultured for 4 days in the second culture medium.

In the provided methods, the culturing and harvesting steps are repeated a number of times. For example, the culturing and harvesting steps can be repeated 3, 4, 5, 6, 7, 8, 9, 10, 11, 12, 13, 14, 15, 16, 17, 18, 19, 20, 21, 22, 23, 24, 25, 26, 27, 28, 29 or 30 times. Optionally, the culturing and harvesting steps (d) and (e) are repeated 4-25 times in fourth to twenty-fifth culture media.

Any of a variety of media are suitable for use in culturing the microorganisms described herein. Optionally, the medium supplies various nutritional components, including a carbon source and a nitrogen source, for the microorganism. Thus, optionally, one or more of the culture media further comprise glucose. For example, one or more of the first, second, third, fourth, fifth, sixth, seventh, etc., culture medium may further include glucose.

When the medium comprises multiple carbon sources, the carbon sources can be provided at particular concentration ratios. For example, the concentration ratio of glucose to xylose one or more of the culture media can be from 2:2 to 2:5 or any ratio between 2:2 to 2:5. Optionally, one or more of the culture media comprise 1, 2, 3, 4, 5, 6, 7, 8, 9, or 10% xylose weight/volume. Optionally, the one or more of the culture media comprises 5% xylose weight/volume. Optionally, one or more of the culture media comprises 20 to 200 g/L xylose or any value or range within 20 to 200 g/L xylose.

Optionally, the xylose is hemicellulosic xylose. Typically, hemicellulosic xylose feedstock comprises primarily xylose and some glucose. By way of example, hemicellulosic xylose feedstocks can include 200 to 450 g/L xylose and 20 to 60 g/L glucose.

When the one or more media further include glucose, the glucose can be hemicellulosic glucose. Typically, hemicellulosic feedstocks include primarily glucose and some xylose. By way of example, hemicellulosic glucose feedstocks can include 40 to 100 g/L xylose and 500 to 600 g/L glucose.

Optionally, one or more of the media can include additional carbon sources. Examples of carbon sources include fatty acids (e.g., oleic acid), lipids, glycerols, triglycerols, carbohydrates, polyols, amino sugars, and any kind of biomass or waste stream. Carbohydrates include, but are not limited to, cellulose, hemicellulose, fructose, dextrose, xylose, lactulose, galactose, maltotriose, maltose, lactose, glycogen, gelatin, starch (corn or wheat), acetate, m-inositol (e.g., derived from corn steep liquor), galacturonic acid (e.g., derived from pectin), L-fucose (e.g., derived from galactose), gentiobiose, glucosamine, alpha-D-glucose-1-phosphate (e.g., derived from glucose), cellobiose, dextrin, alpha-cyclodextrin (e.g., derived from starch), and sucrose (e.g., from molasses). Polyols include, but are not limited to, maltitol, erythritol, and adonitol. Amino sugars include, but are not limited to, N-acetyl-D-galactosamine, N-acetyl-D-glucosamine, and N-acetyl-beta-D-mannosamine.

Also provided are a population of isolated microorganisms made by the provided methods of making microorganisms with increased xylose consumption. The population of microorganisms made from the provided methods are more capable of using hemicellulosic feedstocks their parental counterparts, e.g., control xylose-consuming microorganisms. The population of microorganisms can consume at least 2 g/L/h hemicellulosic xylose in culture medium comprising hemicellulosic xylose as the sole carbon source. Optionally, the population of microorganisms can consume at least 3 g/L/h hemicellulosic xylose in culture medium comprising hemicellulosic xylose and hemicellulosic glucose. Optionally, the population of microorganisms have decreased xylitol production compared to control xylose-consuming microorganisms. Optionally, the population of microorganisms comprises 3, 4, 5, or 6 copies of a xylose kinase, which can be pirXK or any other suitable xylose kinase.

As described, the isolated microorganisms produced by the methods of making microorganisms with increased xylose consumption rate provided herein can be cultured under conditions that produce a compound of interest, e.g., fatty acids, or a specific fatty acid at a desired level. The culturing can be carried out for one to several days. Optionally, the method further includes extracting the oils from the microorganisms. The provided methods include or can be used in conjunction with additional steps for culturing microorganisms according to methods known in the art, obtaining the oils therefrom, or further refining the oil.

Provided is a method of growing isolated microorganisms or the population of microorganisms made by the provided method of making microorganisms with increased xylose consumption, i.e., microorganisms with increased xylose consumption. The method includes culturing the microorganisms in a growth medium comprising a glucose:xylose ratio ranging from 1:10 to 1:1 and a high concentration of a nitrogen source.

Also provided are methods of reducing xylitol production in cultures comprising the isolated microorganisms or population of microorganisms made by the provided method of making microorganisms with increased xylose consumption. The methods include culturing the isolated microorganisms in a growth medium comprising a carbon source and a high concentration of a nitrogen source. Optionally, the carbon source comprises glucose and xylose. Optionally, the growth medium comprises a glucose:xylose ratio ranging from 1:10 to 1:1. Optionally, the glucose is hemicellulosic glucose and the xylose is hemicellulosic xylose.

The culture medium can include 20 to 200 g/L xylose or any value or range within 20 to 200 g/L xylose. Optionally, the xylose is hemicullulosic xylose. Typically, hemicellulosic xylose feedstock comprises primarily xylose and some glucose. By way of example, hemicellulosic xylose feedstocks can include 200 to 450 g/L xylose and 20 to 60 g/L glucose. Optionally, the glucose can be hemicellulosic glucose. Typically, hemicellulosic feedstocks include primarily glucose and some xylose. By way of example, hemicellulosic glucose feedstocks can include 40 to 100 g/L xylose and 500 to 600 g/L glucose. Thus, the provided method includes culturing the microorganisms in a growth medium comprising hemicellulosic glucose:hemicellulosic xylose at a ratio ranging from 1:10 to 1:1.

As used herein, a high concentration of a nitrogen source means the growth medium comprises at least 30 g/L of the nitrogen source. Optionally, the growth medium comprises 20 to 40 g/L of the nitrogen source or 30 to 40 g/L of the nitrogen source. The medium can include any of a variety of nitrogen sources. Exemplary nitrogen sources include ammonium solutions (e.g., NH₄ in H₂O), ammonium or amine salts (e.g., (NH₄)₂SO₄, (NH₄)₃PO₄, NH₄NO₃, NH₄OOCH₂CH₃ (NH₄Ac)), peptone, tryptone, yeast extract, malt extract, fish meal, sodium glutamate, soy extract, casamino acids and distiller grains. Optionally, the nitrogen source is ammonium sulfate.

Optionally, the hemicellulosic carbon source is not pretreated. As used herein, the terms pretreat, pretreated, or pretreatment refers to the removal of impurities that could physically or biologically impact the culture growth. Examples of pretreatment include chemical treatment to precipitate and remove impurities, pH adjustment to match the pH of the culture environment, filtration or centrifugation to remove suspended solid.

Optionally, one or more of the media can include additional carbon sources as described herein.

One or more of the culture media used herein including in the methods of making microorganisms with increased xylose consumption and in the methods of culturing and reducing xylitol consumption in the population of microorganisms made by the provided methods can include saline or salt. The selected culture medium optionally includes NaCl, natural or artificial sea salt, and/or artificial seawater. Thraustochytrids can be cultured, for example, in medium having a salt concentration from about 0.5 g/L to about 50.0 g/L, from about 0.5 g/L to about 35 g/L, or from about 18 g/L to about 35 g/L. Optionally, the Thraustochytrids described herein can be grown in low salt conditions (e.g., salt concentrations from about 0.5 g/L to about 20 g/L or from about 0.5 g/L to about 15 g/L).

Alternatively, the culture medium can include non-chloride-containing sodium salts as a source of sodium, with or without NaCl. Examples of non-chloride sodium salts suitable for use in accordance with the present methods include, but are not limited to, soda ash (a mixture of sodium carbonate and sodium oxide), sodium carbonate, sodium bicarbonate, sodium sulfate, and mixtures thereof. Sec, e.g., U.S. Pat. Nos. 5,340,742 and 6,607,900, which are fully incorporated by reference herein. A significant portion of the total sodium, for example, can be supplied by non-chloride salts such that less than about 100%, 75%, 50%, or 25% of the total sodium in culture medium is sodium chloride.

The medium optionally includes a phosphate, such as potassium phosphate or sodium-phosphate. Inorganic salts and trace nutrients in medium can include ammonium sulfate, sodium bicarbonate, sodium orthovanadate, potassium chromate, sodium molybdate, selenous acid, nickel sulfate, copper sulfate, zinc sulfate, cobalt chloride, iron chloride, manganese chloride calcium chloride, and EDTA. Vitamins such as pyridoxine hydrochloride, thiamine hydrochloride, calcium pantothenate, p-aminobenzoic acid, riboflavin, nicotinic acid, biotin, folic acid and vitamin B12 can be included.

The pH of the medium can be adjusted to between and including 3.0 and 10.0 using acid or base, where appropriate, and/or using the nitrogen source. Optionally, the medium can be sterilized.

Generally a medium used for culture of a microorganism is a liquid medium. However, the medium used for culture of a microorganism can be a solid medium. In addition to carbon and nitrogen sources as discussed herein, a solid medium can contain one or more components (e.g., agar or agarose) that provide structural support and/or allow the medium to be in solid form.

Resulting biomass produced from culturing the isolated microorganisms or population of microorganisms made by the provided methods can be pasteurized to inactivate undesirable substances present in the biomass. For example, the biomass can be pasteurized to inactivate compound-degrading substances, such as degradative enzymes. The biomass can be present in the fermentation medium or isolated from the fermentation medium for the pasteurization step. The pasteurization step can be performed by heating the biomass and/or fermentation medium to an elevated temperature. For example, the biomass and/or fermentation medium can be heated to a temperature from about 50° C. to about 95° C. (e.g., from about 55° C. to about 90° C. or from about 65° C. to about 80° C.). Optionally, the biomass and/or fermentation medium can be heated from about 30 minutes to about 120 minutes (e.g., from about 45 minutes to about 90 minutes, or from about 55 minutes to about 75 minutes). The pasteurization can be performed using a suitable heating means, such as, for example, by direct steam injection.

The biomass can be harvested according to a variety of methods, including those currently known to one skilled in the art. For example, the biomass can be collected from the fermentation medium using, for example, centrifugation (e.g., with a solid-ejecting centrifuge) and/or filtration (e.g., cross-flow filtration). Optionally, the harvesting step includes use of a precipitation agent for the accelerated collection of cellular biomass (e.g., sodium phosphate or calcium chloride).

The biomass is optionally washed with water. The biomass can be concentrated up to about 20% solids. For example, the biomass can be concentrated from about 1% to about 20% solids, from about 5% to about 20%, from about 7.5% to about 15% solids, or to any percentage within the recited ranges.

After biomass processing, oils can be extracted from the isolated microorganisms or population of microorganisms made by the provided methods. Optionally, the oils can be further processed, e.g., by winterization. Prior to winterization, the oils or polyunsaturated fatty acids are obtained or extracted from the biomass or microorganisms using one or more of a variety of methods, including those currently known to one of skill in the art. For example, methods of isolating oils or polyunsaturated fatty acids are described in U.S. Pat. No. 8,163,515, which is incorporated by reference herein in its entirety. Alternatively, the oils or polyunsaturated fatty acids are isolated as described in U.S. Publication No. 2015/0176042, which is incorporated by reference herein in its entirety. Optionally, the one or more polyunsaturated fatty acids are selected from the group consisting of alpha linolenic acid, arachidonic acid, docosahexanenoic acid, docosapentaenoic acid, eicosapentaenoic acid, gamma-linolenic acid, linoleic acid, linolenic acid, and combinations thereof.

Oils, lipids or derivatives thereof (e.g., polyunsaturated fatty acids (PUFAs) and other lipids) that are obtained from the provided isolated microorganisms or population of microorganisms can be utilized in any of a variety of applications exploiting their biological, nutritional, or chemical properties. Thus, the oils, lipids or derivatives thereof can be used to produce biofuel. Optionally, the oils, lipids or derivatives thereof, are used in pharmaceuticals, nutraceuticals, food supplements, animal feed additives, cosmetics, and the like.

Optionally, the oils or biomass can be incorporated into a final product (e.g., a food or feed supplement, an infant formula, a pharmaceutical, a fuel, and the like). Optionally, the biomass can be incorporated into animal feed, for example, feed for cows, horses, fish or other animals. Optionally, the oils can be incorporated into nutritional or dietary supplements like vitamins. Suitable food or feed supplements into which the oils or lipids can be incorporated include beverages such as milk, water, sports drinks, energy drinks, teas, and juices; confections such as candies, jellies, and biscuits; fat-containing foods and beverages such as dairy products; processed food products such as soft rice (or porridge); infant formulae; breakfast cereals; or the like.

Optionally, one or more of the oils or compounds therein (e.g., PUFAs) can be incorporated into a nutraceutical or pharmaceutical product. Examples of such nutraceuticals or pharmaceuticals include various types of tablets, capsules, drinkable agents, etc. Optionally, the nutraceutical or pharmaceutical is suitable for topical application or oral applications. Dosage forms can include, for example, capsules, oils, granula, granula subtilac, pulveres, tabellae, pilulae, trochisci, or the like.

The oils or oil portions thereof produced according to the methods described herein can be incorporated into products in combination with any of a variety of other agents. For instance, the oils or biomass can be combined with one or more binders or fillers, chelating agents, pigments, salts, surfactants, moisturizers, viscosity modifiers, thickeners, emollients, fragrances, preservatives, etc., or any combination thereof.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

The examples below are intended to further illustrate certain aspects of the methods and compositions described herein, and are not intended to limit the scope of the claims.

### EXAMPLES

### Example 1. Strain Growth on Laboratory-Grade and Hemicellulosic Carbon Sources

The strains as described in Table 1 were used.

TABLE 1
Strain Description.

| Strain Name | Strain Description |
|---|---|
| Iso-his# 16 | Modified to express xylose isomerase (SEQ ID NO: 2) |
| Strain 7-7 | Modified to express xylose isomerase (SEQ ID NO: 2) and |
| | Xylulose kinase from E. coli (xylB) (SEQ ID NO: 3) |
| Strain 51-7 | Modified to express xylose isomerase (SEQ ID NO: 2) and |
| | xylulose kinase from Piromyces (pirXK) (SEQ ID NO: 6) |
| Gxs1 7-7 | Modified to express xylose isomerase (SEQ ID NO: 2) and |
| | Xylulose kinase from E coli (xylB) (SEQ ID NO: 3) and |
| | xylose transporter from Candida (gxs1) (SEQ ID NO: 11) |
| AspTx 7-7 | Modified to express xylose isomerase (SEQ ID NO: 2) and |
| | Xylulose kinase from E. coli (xylB) (SEQ ID NO: 3) and |
| | xylose transporter from Aspergillus (asptx) (SEQ ID NO: 9) |

Media composition and hemicellulosic carbohydrate stream characteristics used for fermentations are described in Table 2 and Table 3.

TABLE 2
General fermentation medium composition
excluding carbon concentration

| Ingredient | Amount Per Litre | |
|---|---|---|
| Yeast extract | 2 | g |
| MgSO₄•7H₂O | 4 | g |
| FeCl₃•6H₂O | 0.5 | mL |
| Trace element solution | 1.5 | mL |
| NaCl | 1.65 | g |
| (NH₄)₂SO₄ | 20 | g |
| KH₂PO₄ | 2.2 | g |
| K₂HPO₄ | 2.4 | g |
| CaCl₂•2H₂O | 0.5 | mL |
| Vitamin solution | 1 | mL |

TABLE 3
Composition of xylose and glucose hemicellulosic feedstocks

| | Hemicellulosic | Hemicellulosic |
|---|---|---|
| | xylose | glucose |
| Xylose Concentration (g L⁻¹) | 249-403 | 49-90 |
| Glucose Concentration (g L⁻¹) | 24-55 | 523-543 |
| Acetic Acid (g kg⁻¹) | 5.93-6.54 | 2.06-3.15 |
| Glycolic Acid (g kg⁻¹) | 6.24 | 15.39-21.02 |
| Lactic Acid (g kg⁻¹) | 8.58 | <DL* |
| Levulinic Acid (g kg⁻¹) | 4.14-5.08 | 10.20-10.99 |
| Formic Acid (g kg⁻¹) | <DL*-4.59 | 9.44-9.73 |
| Furans (HMF + Furfurals) (g kg⁻¹) | 2.42 | 2.92-3.99 |

*DL = detection limit

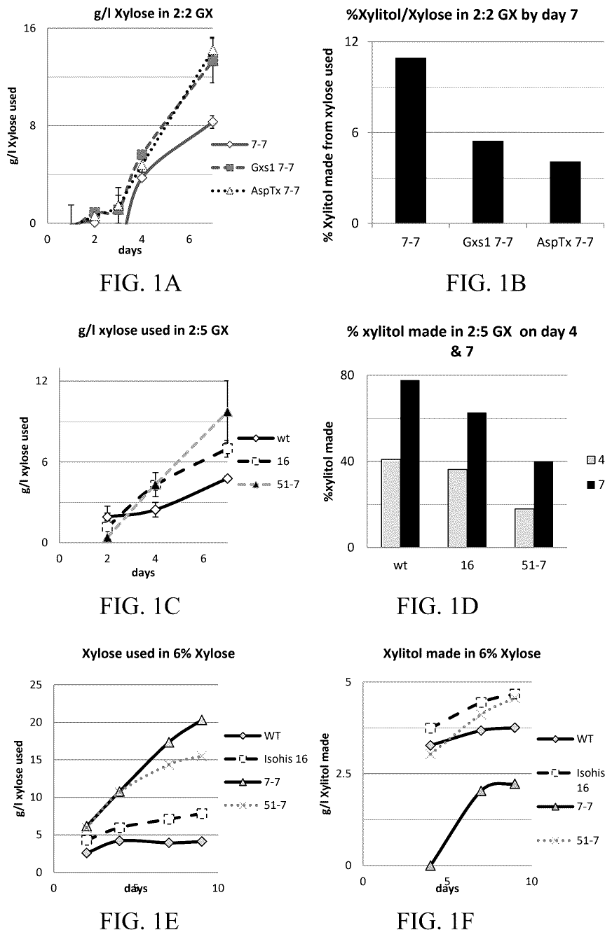

The ability of 7-7, Gxs1 7-7, AspTx 7-7, and 51-7 to metabolize laboratory-grade (not hemicellulosic) xylose was examined by xylose depletion assays (FIG. 1). These flask fermentations demonstrate the ability to metabolize xylose and quantify the amount of xylose converted to xylitol, which can inhibit growth. FIGS. 1A-1F show an increase in xylose metabolism and reduction in xylose converted to xylitol in all strains compared with wild type cells (ONC-T18) not transformed with any genes involved in xylose metabolism.

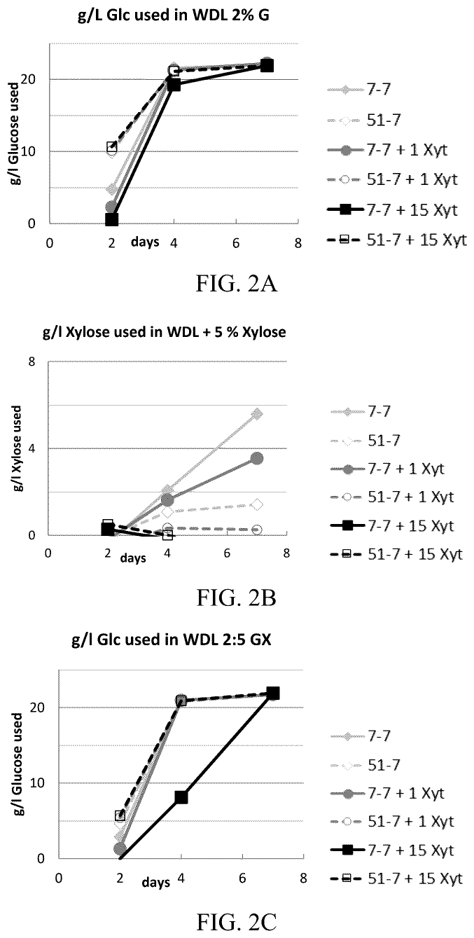

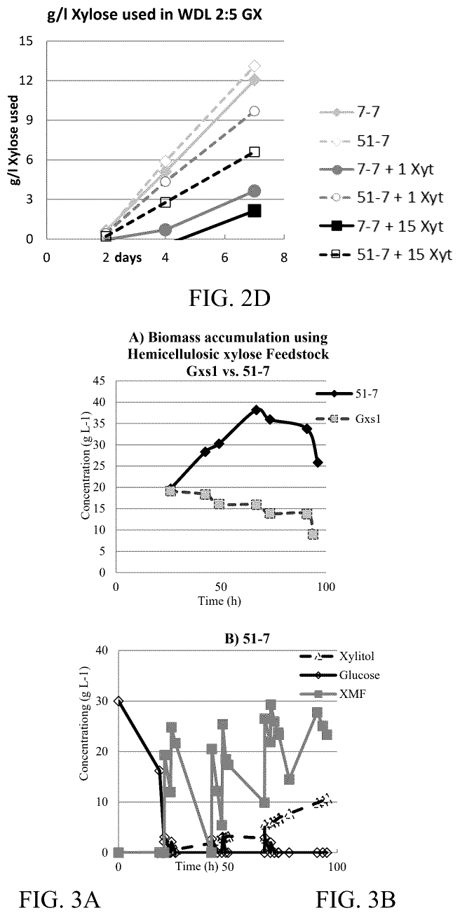

The impact of xylitol concentration on glucose and xylose consumption was determined using 7-7, Gxs1 7-7, and 51-7 strains. These assays indicated that, ideally, xylitol concentrations should be kept lower than about 1 g/L (FIGS. 2A-2D and Table 4), in order to avoid growth inhibition.

TABLE 4
Xylose used, glucose used, and biomass at different
concentrations of xylitol with Gsx1 7-7.

| | Biomass | Glucose used | Xylose |
|---|---|---|---|
| | (g L⁻¹) | (%/g) @48 hr | used (%/g) |
| No Xylitol | 7.450 | 100.0/9.12 | 82.1/16.20 |
| 1 g L⁻¹ Xylitol | 7.225 | 99.9/9.04 | 56.2/11.03 |
| 5 g L⁻¹ Xylitol | 6.683 | 43.7/3.99 | 25.5/5.06 |
| 10 g L⁻¹ Xylitol | 6.425 | 18.6/1.66 | 22.0/4.24 |
| 15 g L⁻¹ Xylitol | 6.592 | 12.8/1.13 | 19.3/3.67 |

In xylose depletion flask assays, Gxs1 7-7 was used with medium containing 20 g/L xylose and 8 g/L glucose and spiked with increasing concentrations of xylitol as shown in Table 4. The values in Table 4 are the total amount of biomass produced and xylose used after 144 hours. The glucose used amounts are shown at 48 hours.

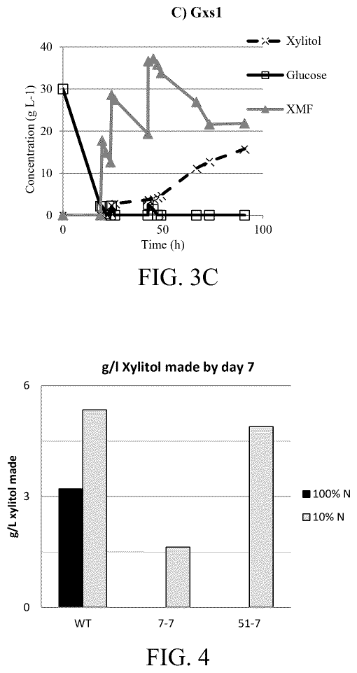

Fermentation assays with 51-7 and Gxs1 7-7 showed similar xylitol constraints when grown in hemicellulosic xylose (FIGS. 3A, 3B and 3C, Table 5). The performance of 51-7 and Gxs1 7-7 was investigated using a hemicellulosic xylose feedstock at a 2 L batch-fed scale. Cells were grown for 72 hours in media as described in Table 4 and batched with 60 g/L glucose. After 72 hours, fermentors were filled with 900 mL of media as described in Table 4 and sterilized by autoclaving. Once fermentor vessels were cooled, 100 mL of prepared cell culture was added, and inoculated vessels were fed with hemicellulosic xylose feedstock. Feeds were kept lower than 30 g/L and continued based on xylose consumption rates. The agitation was increased from 500-1000 RPM throughout fermentation to ensure the maximum consumption rate was reached for both strains. Sampling was performed twice a day to monitor growth and carbon consumption using HPLC. Results and fermentation parameters are summarized in Table 5.

TABLE 5
Fermentation parameters of 2 L scaled batch-fed fermentations
with 51-7 and Gxs1 7-7 with hemicellulosic xylose

| Strain: | 51-7 | Gxs1 7-7 |
|---|---|---|
| Scale (L): | 2 | |
| Agitation (RPM): | 500-1000 | |
| Batch: | 30 g L⁻¹ Glucose | 30 g L⁻¹ Glucose |
| Feedstock Composition: | 403 g L⁻¹ Xylose | 403 g L⁻¹ Xylose |
| | 55 g L⁻¹ Glucose | 55 g L⁻¹ Glucose |
| Target Feeding: | 1 L of | 1 L of |
| | hemicellulosic | hemicellulosic |
| | xylose | xylose |
| Average Carbon Consumed: | 96 g Xylose | 36 g Xylose |
| | 44 g Glucose | 8 g Glucose |
| Xylitol Accumulation (g): | 7 | 13 |
| Average Final Biomass (g L⁻¹): | 38 | 15 |
| Peak Xylose Consumption | 3.30 | 1.23 |
| Rate (g L⁻¹ h⁻¹): | | |
| Average Xylose Consumption | 2.8 | 0.78 |
| Rate (g L⁻¹ h⁻¹): | | |
| Fermentation Length (h): | 96 | 93 |
| Total Fatty Acid Content | 209 | 224 |
| (mg g⁻¹): | | |

Low nitrogen concentration in the medium was shown in flasks assays to correlate with increased xylitol production by the parental strain and 51-7 (FIG. 4) indicating that the nitrogen concentration in the media should be increased in keep xylitol production low.

The abilities of 7-7, Gxs1 7-7, and AspTx 7-7 to grow in medium containing either laboratory-grade, or hemicellulosic carbon sources were further tested in flask fermentations. Strains were grown in flasks containing hemicellulosic xylose. Alternatively, the strains were grown in laboratory-grade xylose and glucose at a ratio of 1:10 mimicking the ratio in a hemicellulosic xylose feedstock. In medium composed of the laboratory-grade carbon stream 7-7, Gxs1 7-7 and AspTx 7-7 used about 6-8 times more xylose than wild-type parental strain (ONC-T18). However, in medium with hemicellulosic xylose, 7-7 did not consume xylose, while Gxs1 7-7 and AspTx 7-7 consumed 82% and 44%, respectively, of available xylose. The results are shown in Table 6.

TABLE 6
Xylose metabolized and xylitol produced in flask assays with
WT, 7-7, Gxs1 7-7, and AspTx 7-7 in medium containing either
laboratory-grade carbon source or hemicellulosic xylose.

| | Xylose used (%/g) | | Xylitol produced (g L⁻¹) | |
|---|---|---|---|---|
| | Laboratory- | | Laboratory- | |
| | grade | | grade | |
| | Glucose: | Hemicellulosic | Glucose: | Hemicellulosic |
| Strain | Xylose | xylose | Xylose | xylose |
| WT | 6.2/1.18 | 7.5/1.57 | 0.901 | 0.000 |
| 7-7 | 51.2/9.73 | 0.0/0.00 | 0.865 | 0.000 |
| Gxs1 7-7 | 36.7/6.98 | 82.0/17.18 | 1.429 | 1.679 |
| AspTx 7-7 | 58.9/11.20 | 44.5/9.32 | 1.030 | 1.094 |

In medium containing laboratory-grade 1:10 glucose:xylose, 7-7, Gxs1 7-7 and AspTx 7-7 used approximately 6 to 9.5 times more xylose than wild-type parental strain. In medium with hemicellulosic xylose, 7-7 did not consume xylose, whereas Gxs1 7-7 and AspTx 7-7 consumed 11 times and 6 times, respectively, more xylose than wild-type. These flask assays also showed that Gxs1 7-7 and AspTx 7-7 had different abilities to use xylose depending on whether the medium contained a laboratory-grade carbon source or hemicellulosic carbon source. For example, although AspTx 7-7 used 1.6× more xylose than Gxs1 7-7 in medium with a laboratory-grade carbon source, Gxs1 7-7 used 1.8× more xylose than AspTx 7-7 in medium containing a hemicellulosic carbon source. This differential usage depending on the source of the carbon implies strains could be optimized for specific carbon sources (Table 6).

Preferred glucose:xylose ratios were also determined in flask experiments. Strains were grown in medium containing different ratios of glucose:xylose for 7 to 9 days, in duplicate for each treatment. Samples were taken every 2 days and centrifuged, the supernatant was collected for HPLC analysis to determine the concentrations of xylose, glucose and xylitol, and the pellets were dried for biomass determination. The results are shown in Table 7.

TABLE 7
Testing different carbon source ratios for impact on xylose
usage and xylitol production with 7-7 and Gxs1 7-7 strains

| | | Laboratory-grade Glc/Xyl | | | Hemicellulosic xylose | | |
|---|---|---|---|---|---|---|---|
| | | | Xylose | Xylitol | | Xylose | Xylitol |
| | | | used | Produced | | used | Produced |
| Glc:Xyl | | Biomass | (%/g | (g/L | Biomass | (% range/ | (g/L |
| ratios | Strain | (g/L) | range) | range) | (g/L) | g range) | range) |
| 1 g/L | 7-7 | — | —/— | —/— | 1.70-4.45 | 49.5-100.0/ | 0.52-1.33 |
| Glc + | | | | | | 5.49-11.44 | |
| 10 g/L | Gxs1 | — | —/— | —/— | 2.35-3.81 | 68.0-100.0/ | 0.00-1.39 |
| Xyl | 7-7 | | | | | 7.55-11.44 | |
| 2 g/L | 7-7 | — | —/— | —/— | 0.90-5.09 | 27.5-65.4/ | 0.00-1.33 |
| Glc + | | | | | | 5.73-14.80 | |
| 20 g/L | Gxs1 | — | —/— | —/— | 0.93-5.16 | 32.1-100.0/ | 0.00-0.86 |
| Xyl | 7-7 | | | | | 6.71-22.62 | |
| 3 g/L | 7-7 | — | —/— | —/— | 1.03-3.20 | 10.7-47.0/ | 0 |
| Glc + | | | | | | 3.81-13.29 | |
| 30 g/L | Gxs1 | — | —/— | —/— | 1.00-4.53 | 10.5-88.5/ | 0 |
| Xyl | 7-7 | | | | | 3.75-25.01 | |
| 10 g/L | 7-7 | 7.43 | 91.2/ | 0.68-1.44 | 3.88-7.43 | 56.6-100.0/ | 0.57 |
| Glc + | | | 12.46 | | | 6.38-13.25 | |
| 10 g/L | Gxs1 | 8.99 | 100.0/ | 0.00-1.22 | 4.53-7.36 | 72.4-100.0/ | 0.16 |
| Xly | 7-7 | | 13.81 | | | 8.16-13.31 | |
| 20 g/L | Gxs1 | 13.95 | 78.5/22.61 | 0 | 11.38 | 64.3/17.83 | 0 |
| Glc + | 7-7 | | | | | | |
| 20 g/L | | | | | | | |
| Xyl | | | | | | | |
| 30 g/L | Gxs1 | 17.33 | 56.5/25.06 | 0 | 1.8 | 0.0/0.00 | 0 |
| Glc + | 7-7 | | | | | | |
| 30 g/L | | | | | | | |
| Xyl | | | | | | | |

Using laboratory-grade glucose:xylose and carbohydrates derived from xylose 5 hemicellulosic stocks, increased xylose consumption occurred when glucose:xylose ratios ranged from about 1:10 to 1:1. Hemicellulosic xylose in a concentration range of about 20 g/L to 30 g/L was preferred for biomass production or xylose consumption (Table 7).

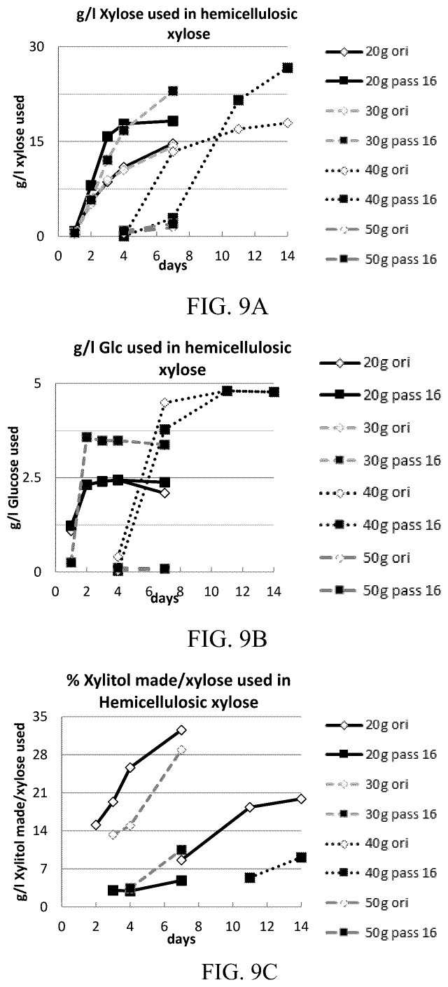

Increasing hemicellulosic xylose concentrations were also tested. Cells were grown in medium for 2 to 3 days. Pellets were washed twice in 9 g/L saline. Then, medium containing 20 g/L, 30 g/L, 40 g/L, or 50 g/L of hemicellulosic xylose was inoculated to an OD600 of 0.5 with the washed cells. Samples were taken at various time points, and the amount of carbohydrate remaining in the supernatant was analyzed by HPLC. In media containing hemicellulosic xylose, 51-7 XP16 used 1.2× to 8.8× more xylose than the original 51-7 strain depending on the amount of hemicellulosic xylose in the media (FIG. 9).

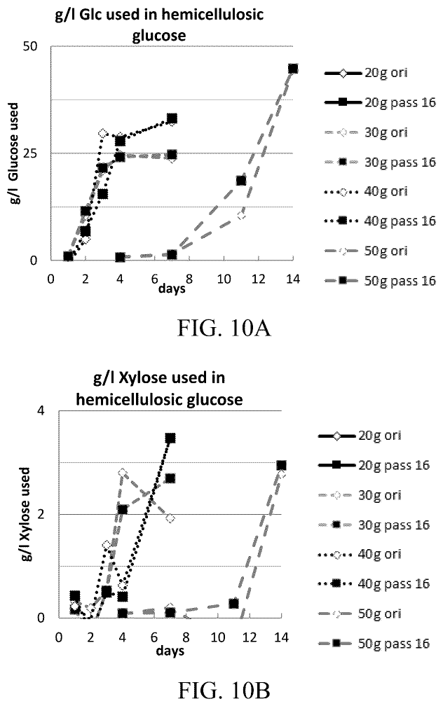

The ability of strains to use hemicellulosic glucose was also tested. The composition of hemicellulosic glucose is in Table 5. Cells were grown in medium for 2 to 3 days. Pellets were washed twice in 9 g/L saline. Then, medium containing 30 g/L, 40 g/L, or 50 g/L of hemicellulosic glucose was inoculated to an OD600 of 0.5 with the washed cells. Samples were taken at various time points, and the amount of carbohydrate remaining in the supernatant was analyzed by HPLC. This passaged strain's ability to use glucose in media containing hemicellulosic glucose was not hindered (FIG. 10).

### Example 2. Strain Adaptation to Improve Xylose Consumption

Strains 7-7, AspTx 7-7 and 51-7 were used to improve xylose consumption by passaging the strains in either medium containing xylose as sole carbon source or medium containing both glucose and xylose. Specifically, strain passaging was performed by culturing the strains in medium containing 50 g/L xylose with or without 20 g/L glucose for 3 to 7 days, removing a portion of the culture, adding the portion to fresh medium and repeating this process multiple times. Each round of passaging included culturing the strains in medium containing 50 g/L xylose with or without 20 g/L glucose for 3 to 7 days after which a portion of the culture was removed and the portion was added to fresh medium. The strains were passaged as many as 22 times. Glycerol stocks were made at each passage to preserve each stage.

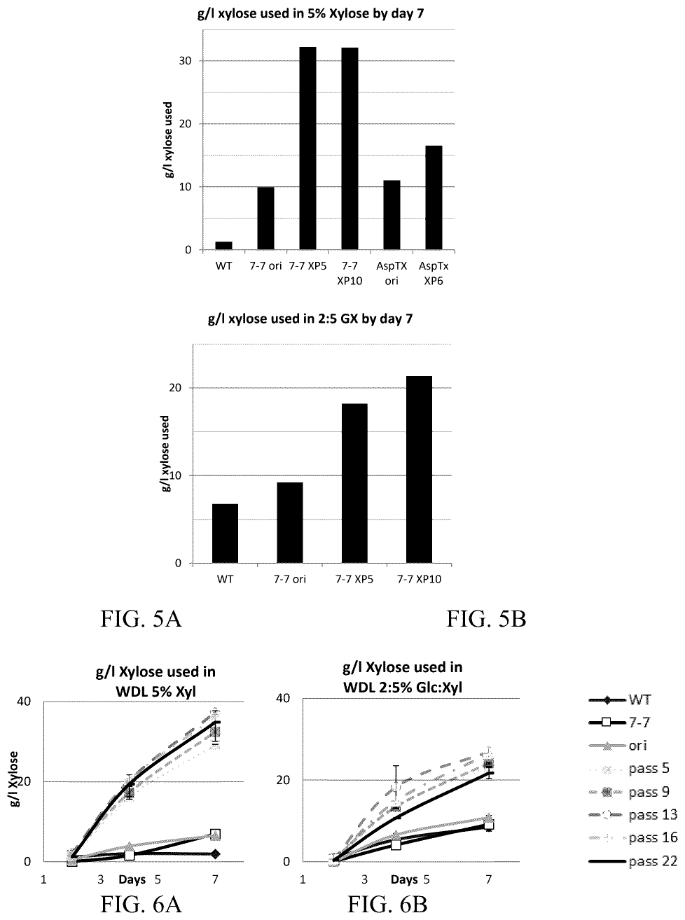

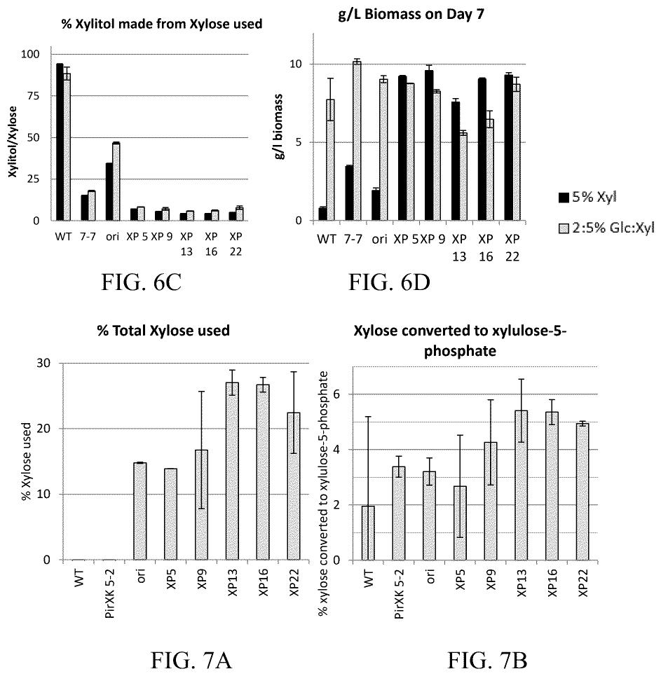

To test xylose consumption of passaged strains, cells were grown in medium for 2 to 3 days and pelleted. Pellets were washed twice in 9 g/L saline. Then, medium containing 50 g/L xylose (5% xylose) or medium containing 20 g/L glucose and 50 g/L xylose (2:5 Glucose:Xylose) was inoculated to an OD600 of 0.05 with the washed cells. Samples were taken at various time points, and the amount of carbohydrate remaining in the supernatant was analyzed by HPLC. The results are shown in FIGS. 5A and 5B. The parental strain, Iso-His #16, did not improve following the laboratory adaptation protocol. However, passaging of 7-7 and AspTx 7-7 resulted in strains with increased xylose usage (FIGS. 5A and 5B). FIGS. 6A, 6B, 6C and 6D, show improvement in xylose usage and xylitol production by 51-7 passaged strains when grown in various media containing either 2:5 Glcose:Xylose or 5% xylose (i.e., 50 g/L xylose). Improvement in xylose usage by strains passaged from 5 to 22 times ranged from 1.5× to 5.5× compared to the unpassaged, parental strain. (FIGS. 5 and 6).

Cell extracts were taken from the passaged strains to analyze enzyme activity. Cell extracts on these passaged strains showed that the strains had higher enzymatic activities for the xylose isomerase and xylulose kinases. FIGS. 7A and 7B show the data for 51-7 passaged strains (51-7 XP5, 51-7 XP9, 51-7 XP13, 51-7 XP16 and 51-7 XP22).

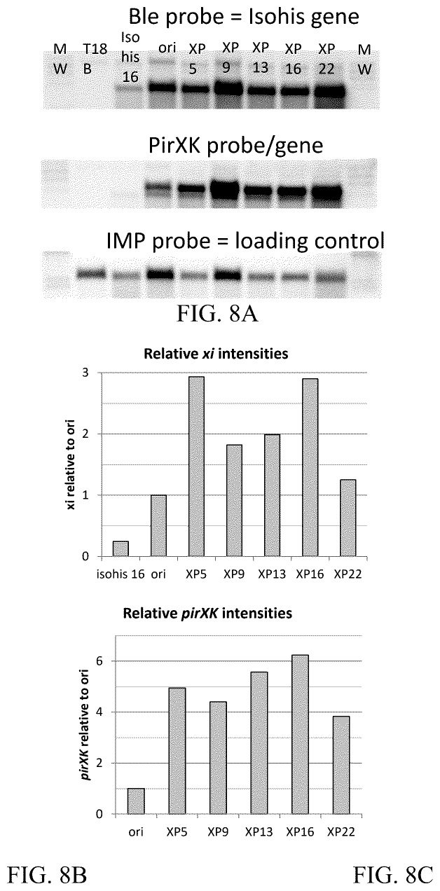

To analyze whether gene copy number changes contribute to the differences in xylose consumption, Southern blot analyses were performed on the 51-7 original (unpassaged) and 51-7 passaged strains. Southern blots were performed using standard protocols. The band signal intensities were normalized to the loading control signal (IMP) then the relative intensities of the xylose isomerase gene (xi) and the pirXK bands in the passaged strains and that of the 51-7 original (ori) genes were calculated. No change was seen in the banding pattern between 51-7 original (referred to in FIG. 8 as ori) and the passaged strains by Southern blotting. However, the intensity of the bands did change relative to the loading control indicating potentially increased copy numbers of xylose isomerase and xylulose kinase (FIG. 8).

### Example 3. Analysis of 51-7 XP16 Adapted Strain

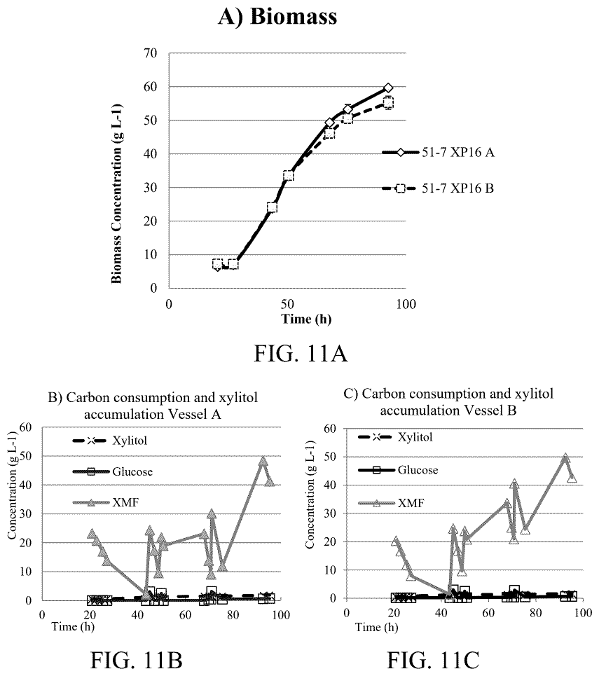

Duplicate 2 L batch-fed fermentations using 51-7 XP16 were performed using 30 g/L laboratory-grade xylose followed by feeding with a feedstock of laboratory-grade xylose and glucose in proportions similar to a hemicellulosic xylose feedstock as described in Table 5. Feeds were generally kept lower than 30 g L-1 xylose; however, 51-7 XP16 in both vessels had greater than 30 g/L xylose concentration after about 70 hours in both vessels due to a decrease in xylose consumption rate. Overall, the average final biomass concentration was 57 g/L (FIG. 11A) at 93 hours. Strain 51-7 XP16 had an average xylose consumption rate of 3.62 g/L/h and a peak xylose consumption rate of 4.99 g/L/h (FIG. 11B). Fermentation results and additional details are outlined in Table 8.

TABLE 8
Fermentation parameters and averaged results of
scaled batch-fed fermentation with 51-7 XP16
and laboratory-grade xylose-glucose feedstock

| Strain: | 51-7 XP16 | |
|---|---|---|
| Scale: | 2 | L |
| Batch: | 30 g L⁻¹ Xylose | |
| Agitation: | 500-1000 | RPM |

| | |
|---|---|
| Feedstock Composition: | 400 g L⁻¹ Xylose |
| | 55 g L⁻¹ Glucose |
| Target Feeding: | 1 L of Xylose-Glucose |
| | Feedstock |
| Average Carbon Consumed (g): | 230 g Xylose |
| | 39 g Glucose |
| Xylitol Accumulation (g): | 3 |
| Average Final Biomass (g/L): | 57 |
| Peak Xylose Consumption | 5.0 |
| Rate (g/L/h): | |
| Average Xylose Consumption | 3.6 |
| Rate (g/L/h): | |
| Fermentation Length (h): | 95 |
| Total Fatty Acid Content | 545 |
| (mg/g): | |

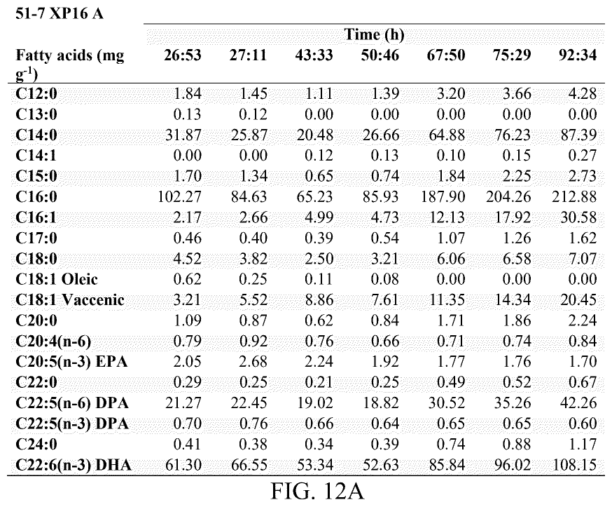

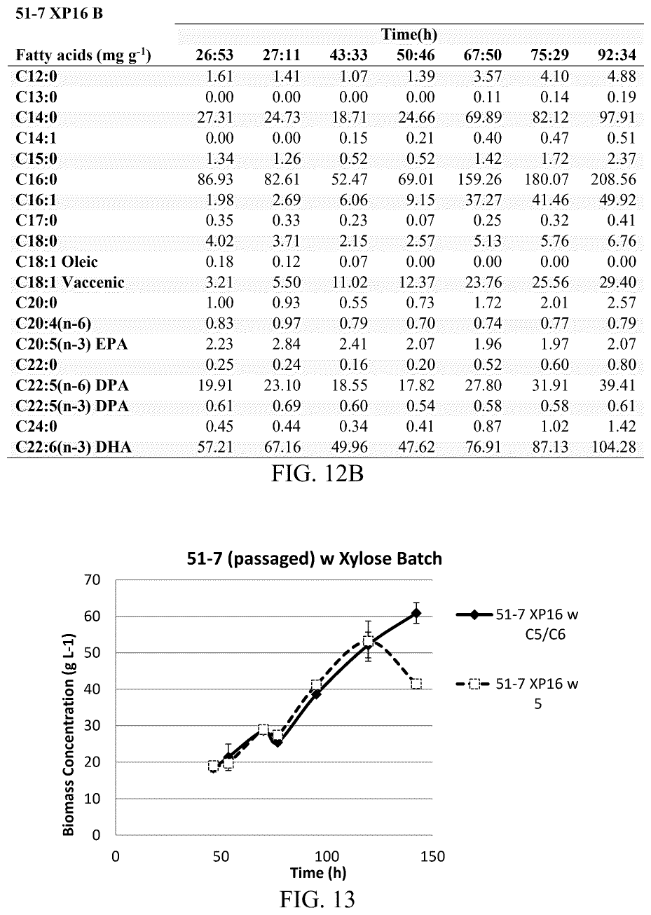

Overall, the 51-7 XP16 strain using laboratory-grade xylose and glucose at concentrations similar to hemicellulosic xylose streams resulted in an average xylose rate of 3.6 g/L/h with 57 g/L final biomass and 545 mg/g total fatty acid content. The fatty acid profile of the strain is shown in FIG. 12.

51-7 XP16's ability to grow on hemicellulosic carbon sources and different concentrations of nitrogen was then analyzed. Fermentation in the presence of increased nitrogen source (40 g/L) was shown to improve hemicellulosic xylose usage. (FIG. 13 and Table 9). Improving the performance of 51-7 was investigated by doubling the concentration of the nitrogen source to 40 g/L from the original concentration shown in Table 4. Increasing biomass accumulation was also investigated by switching the feedstock to hemicellulosic glucose during nitrogen depletion for one of the vessels. Cells were grown for 72 hours in media as described in Table 4 and batched with 60 g/L of glucose. After 72 hours the fermentors were filled with 900 mL of media as described in Table 4 and sterilized by autoclaving. Once the fermentor vessels were cooled, 100 mL of prepared cell culture was added. The fermentors were batched with laboratory-grade xylose and fed with a hemicellulosic feedstock as described in Table 9. The composition of the media is described in Table 4. Feeds for vessel #1 were kept lower than 30 g/L xylose and continued based on xylose consumption rates. Vessel #2 was fed similarly, except the feedstock was switched once nitrogen was deleted. The agitation was increased from 500-1000 RPM throughout the fermentations to ensure the maximum consumption rate was reached. In the end, 51-7 with double nitrogen (40 g/L (NH₄)₂SO₄) outperforms 51-7 in regular media (20 g/L (NH₄)₂SO₄), and the growth can continue if the feedstock is switched to a glucose-type feedstock at nitrogen depletion.

TABLE 9
Fermentation parameters of 2 L scaled batch-fed fermentations
with 51-7 XP16 with hemicellulosic xylose

| Vessel: | Vessel #1 | Vessel #2 |
|---|---|---|
| Strain: | 51-7 XP16 | 51-7 XP 16 |
| Feedstock: | hemicellulosic | hemicellulosic |
| | xylose + | xylose |
| | hemicellulosic | |
| | glucose | |
| Scale (L): | 2 L | |
| Agitation (RPM): | 500-1000 | |
| Batch: | 30 g/L Xylose | 30 g L⁻¹ Xylose |
| Feedstock Composition: | 403 g/L Xylose/ | 403 g L⁻¹ Xylose |
| | 55 g/L Glucose + | 55 g L⁻¹ Glucose |
| | 543 g/L Glucose | |
| | 49 g/L Xylose | |
| Target Feeding: | 0.5 L of | 1 L of |
| | hemicellulosic | hemicellulosic |
| | xylose + | xylose |
| | 0.5 L of | |
| | hemicellulosic | |
| | glucose | |
| Average Carbon | 173 g Xylose | 287 g Xylose |
| Consumed: | 226 g Glucose | 38 g Glucose |
| Xylitol Accumulation (g): | 12 | 11 |
| Average Final Biomass | 61 | 53 |
| (g/L): | | |
| Peak Xylose Consumption | 3.0 | 2.2 |
| Rate (g/L/h): | | |
| Average Xylose | 2.2 | 1.6 |
| Consumption Rate | | |
| (g/L/h): | | |
| Fermentation Length (h): | 142 | 119 |
| Total Fatty Acid Content | 300 | 148 |
| (mg/g): | | |

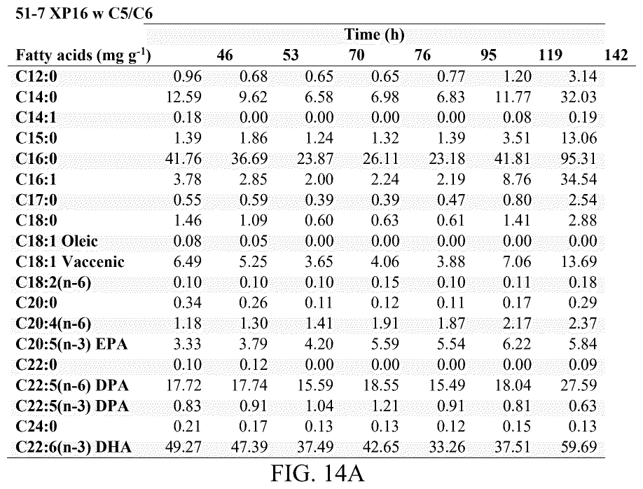

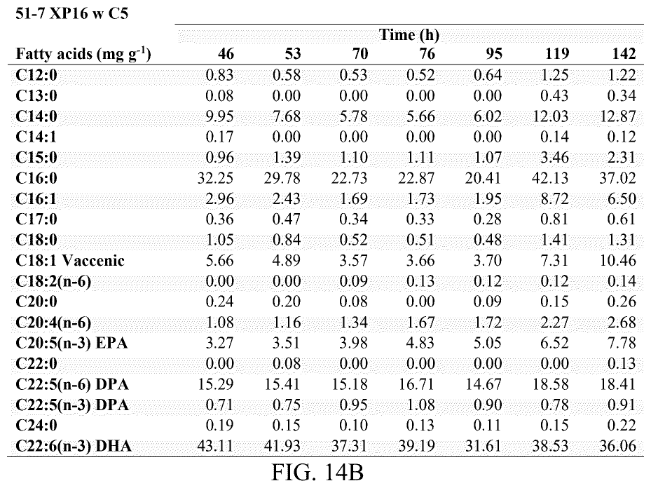

Although the average xylose consumption ranged from 1.6 to 2.3 g/L/h, there was an increase in the total xylose consumed compared to consumption of laboratory-grade xylose (287 g xylose versus 230 g xylose, respectively) by 51-7 XP16. The fatty acid profiles are shown in FIG. 14.

The ability of 51-7 XP16 to grow on hemicellulosic glucose streams efficiently was also demonstrated at 5 L, 30 L and 3200 L volumes. Fermentations were batched with 30 g/L glucose and fed with hemicellulosic glucose blended with laboratory-grade glucose. The 5 L and 30 L vessels were inoculated with cells grown for 72 hours in media, while the 3200 L vessel was inoculated with 100 L of seed grown in a 190 L vessel for 24 hours in medium at 28° C. and pH 5.75. The 5, 30 and 3200 L fermentations were run at 28° C., pH 5.75 and aeration of 1 vvm. The parameters are shown in Table 10.

TABLE 10
Parameters of 51-7 XP fermentations at 5 L, 30 L, and 3200 L with hemicellulosic glucose.

| | | | | Oil | Glucose | Xylose | Xylitol |
|---|---|---|---|---|---|---|---|
| | | | Biomass | content | used | used | produced |
| Scale | | Strain | (g/L) | (mg/g) | (%/Kg) | (%/Kg) | (g/L) |
| 5 | L | 51-7 XP16 | 89 | 763.6 | 100/1.40 | 68.0/0.51 | 1.400 |
| | | Wild type | 112 | 773.7 | 97.9/1.74 | 52.0/0.39 | 1.500 |
| | | [ONC-T18] | | | | | |
| 30 | L | 51-7 XP16 | 100-102 | 728.0-739.0 | 100/7.30-8.00 | 85-88/0.56-0.62 | 0.000-0.000 |
| | | Wild type | — | — | — | — | — |
| 3200 | L | 51-7 XP16 | 124 | 781.4 | 99.3/1076.4 | 51.2/36.4 | 0.000 |

| | | | | | |
|---|---|---|---|---|---|
| Wild type* | 121.2 ± 7.7 | 784.5 ± 13.2 | 99.6 ± 0.42/ | 18.0 ± 14.5/ | 0.000 ± 0.00 |
| | | | 1001.7 ± 52.0 | 8.5 ± 7.0 | |

The 5 L fermentations were run and fed using a 1:1 ratio of laboratory-grade glucose to hemicellulosic glucose, to evaluate the performance of 51-7 XP16 compared to the wild-type strain. The fermentation was finished at 74 hours for both strains with a final biomass of 89 g/L for 51-7XP16 and 112 g/L for the wild-type strain and 763.6 mg/g oil for 51-7 XP16 and 773.7 mg/g oil for wild-type parental strain ONC-T18.

The feeding strategy for the 5 L, 10 L, and the 51-7 XP16 3200 L scales, allowed for glucose starvation to promote the consumption of xylose. The average glucose consumption rate was 10.8 g/L/h, while the average xylose consumption rate was 0.7 g/L/h. The different metabolic rates caused xylose to accumulate up to 11.12 g/L of xylose without extracellular xylitol accumulation. The 3200 L scale was fed continuously, which reduced xylitol production.

Although 51-7 XP16 produced less biomass, at times, compared to wild-type, it metabolized more xylose than the wild-type strain (ONC-T18) and produced less xylitol. 51-7 XP16 used 68% of xylose, producing 1.4 g/L of xylitol while wild-type used 52% of xylose producing 1.5 g/L xylitol.

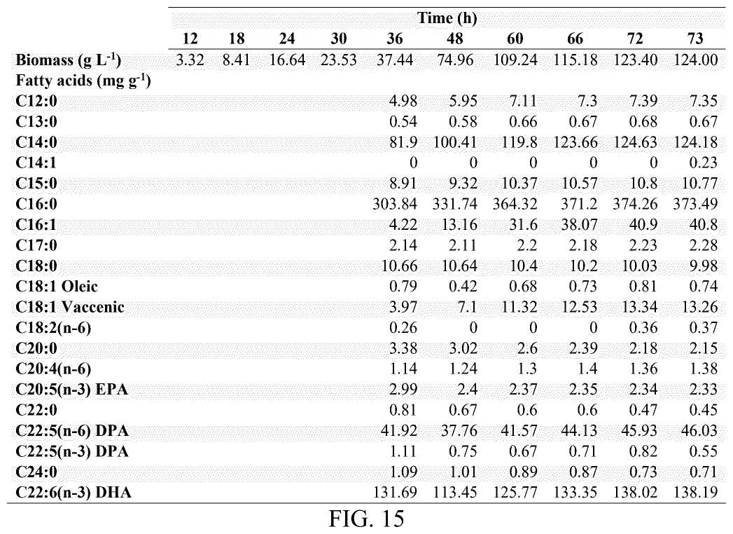

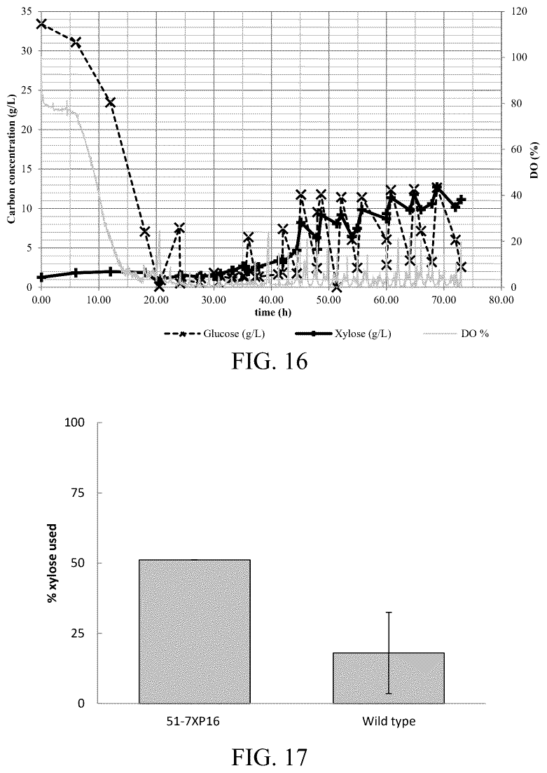

Compared to wild type (ONC-T18) at 5 L, 51-7 XP16 used 1.3 times more xylose. This process was scalable at 30 L and 3200 L with 51-7 XP using 51% of the xylose at 3200 L (FIG. 15, FIG. 16, and FIG. 17).

In comparison with wild-type (ONC-T18), the xylose enhanced strain used up to 51.16% of the xylose fed while avoiding the production of xylitol. Further. 51-7 XP16 used in average 2.8 times more xylose than wild-type.

1. A method of making microorganisms with increased xylose consumption comprising
a. providing xylose-consuming microorganisms comprising two or more copies of a nucleic acid sequence encoding xylose isomerase and two or more copies of a nucleic acid sequence encoding a xylose kinase;
b. culturing the microorganisms in a first culture medium comprising xylose for at least 3 days;
c. harvesting a portion of the microorganisms from the first culture medium after culture step (b);
d. culturing the harvested portion of microorganisms in a second culture medium comprising xylose for at least 3 days;
e. harvesting a portion of the microorganisms from the second culture medium after culture step (d);
f. repeating culturing and harvesting steps (d) and (e) at least two times in a third culture medium and a fourth culture medium;
g. isolating the harvested microorganisms from step (f), wherein the isolated microorganisms have increased xylose consumption rates compared to control xylose-consuming microorganisms.

2. The method of claim 1, wherein one or more of the culture media further comprises glucose.

3. The method of claim 2, wherein the first culture medium further comprises glucose.

4. The method of claim 2, wherein the second culture medium further comprises glucose.

5. The method of claim 4, wherein the concentration ratio of glucose to xylose in the culture medium or culture media is from 2:2 to 2:5.

6. The method of claim 1, wherein the microorganisms are cultured 3 to 7 days in one or more of the culturing steps.

7. The method of claim 1, wherein one or more of the culture media comprise 5% xylose weight/volume.

8. The method of claim 1, wherein one or more of the culture media comprises 20 to 200 g/L xylose.

9. The method of claim 1, wherein the culturing and harvesting steps (d) and (e) are repeated 4-25 times in fourth to twenty-fifth culture media.

10. The method of claim 1, wherein the isolated microorganisms consume at least 2 g/L/h hemicellulosic xylose in culture medium comprising hemicellulosic xylose as the sole carbon source.

11. The method of claim 1, wherein the xylose is hemicellulosic xylose.

12. The method of claim 1, wherein the isolated microorganisms consume at least 3 g/L/h hemicellulosic xylose in culture medium comprising hemicellulosic xylose and hemicellulosic glucose.

13. A population of isolated microorganisms made by the method of claim 1.

14. The population of isolated microorganisms of claim 13, wherein the microorganisms have decreased xylitol production compared to control microorganisms.

15. The population of isolated microorganisms of claim 13, wherein the microorganisms comprise 3, 4, 5, or 6 copies of a xylose kinase.

16. The population of isolated microorganisms of claim 15, wherein the xylose kinase is pirXK.

17. A method of growing the isolated microorganisms made by the method of claim 1 comprising culturing the microorganisms in a growth medium comprising a glucose:xylose ratio ranging from 1:10 to 1:1 and a high concentration of a nitrogen source.

18. The method of claim 17, wherein the growth medium comprises from 20 g/L to 200 g/L xylose.

19. The method of claim 17, wherein the glucose is hemicellulosic glucose and the xylose is hemicellulosic xylose.

20. The method of claim 17, wherein the growth medium comprises at least 30 g/L of the nitrogen source.

21. The method of claim 17, wherein the growth medium comprises 20 to 40 g/L of the nitrogen source.

22. The method of claim 17, wherein the nitrogen source is ammonium sulfate.

23. A method of reducing xylitol production in cultures comprising xylose-consuming microorganisms made by the method of claim 1 comprising culturing the isolated microorganisms in a growth medium comprising a carbon source and a high concentration of a nitrogen source.

24. The method of claim 23, wherein the growth medium comprises at least 30 g/L of the nitrogen source.

25. The method of claim 23, wherein the growth medium comprises 20 to 40 g/L of the nitrogen source.

26. The method of claim 23, wherein the nitrogen source is ammonium sulfate.

27. The method of claim 23, wherein the carbon source comprises a hemicellulosic carbon source.

28. The method of claim 27, wherein the hemicellulosic carbon source is not pretreated.

29. The method of claim 23, wherein the carbon source comprises glucose and xylose.

30. The method of claim 29, wherein the growth medium comprises a glucose:xylose ratio ranging from 1:10 to 1:1.

31. The method of claim 29, wherein the glucose is hemicellulosic glucose and the xylose is hemicellulosic xylose.


SEQUENCE LISTING (12 sequences)

SEQ ID NO: 1 - DNA, 1723 bp, Thraustochytrium sp.
  source 1..1723: mol_type = genomic DNA
gtagtcatacgctcgtctcaaagattaagccatgcatgtgtaagtataagcgattatactgtgagactgcgaacggctcattatatcagttatgatttcttcggtattttctttatatggatacctgcagtaattctggaattaatacatgctgagagggcccgactgttcgggagggccgcacttattagagttgaagccaagtaagatggtgagtcatgataattgagcagatcgcttgtttggagcgatgaatcgtttgagtttctgccccatcagttgtcgacggtagtgtattggactacggtgactataacgggtgacggggagttagggctcgactccggagagggagcctgagagacggctaccacatccaaggaaggcagcaggcgcgtaaattacccaatgtggactccacgaggtagtgacgagaaatatcaatgcggggcgcttcgcgtcttgctattggaatgagagcaatgtaaaaccctcatcgaggatcaactggagggcaagtctggtgccagcagccgcggtaattccagctccagaagcgtatgctaaagttgttgcagttaaaaagctcgtagttgaatttctggggcgggagccccggtctttgcgcgactgcgctctgtttgccgagcggctcctctgccatcctcgcctctttttttagtggcgtcgttcactgtaattaaagcagagtgttccaagcaggtcgtatgacctggatgtttattatgggatgatcagatagggctcgggtgctattttgttggtttgcacatctgagtaatgatgaataggaacagttgggggtattcgtatttaggagctagaggtgaaattcttggatttccgaaagacgaactacagcgaaggcatttaccaagcatgttttcattaatcaagaacgaaagtctggggatcgaagatgattagataccatcgtagtctagaccgtaaacgatgccgacttgcgattgcggggtgtttgtattggaccctcgcagcagcacatgagaaatcaaagtctttgggttccggggggagtatggtcgcaaggctgaaacttaaaggaattgacggaagggcaccaccaggagtggagcctgcggcttaatttgactcaacacgggaaaacttaccaggtccagacataggtaggattgacagattgagagctctttcttgattctatgggtggtggtgcatggccgttcttagttggtggagtgatttgtctggttaattccgttaacgaacgagacctcggcctactaaatagcggtgggtatggcgacatacttgcgtacgcttcttagagggacatgttcggtatacgagcaggaagttcgaggcaataacaggtctgtgatgcccttagatgttctgggccgcacgcgcgctacactgatgggttcaacgggtggtcatcgttgttcgcagcgaggtgctttgccggaaggcatggcaaatcctttcaacgcccatcgtgctggggctagatttttgcaattattaatctccaacgaggaattcctagtaaacgcaagtcatcagcttgcattgaatacgtccctgccctttgtacacaccgcccgtcgcacctaccgattgaacggtccgatgaaaccatgggatgaccttttgagcgtttgttcgcgaggggggtcagaactcgggtgaatcttattgtttagaggaaggtgaagtc

SEQ ID NO: 2 - DNA, 1320 bp, Thraustochytrium sp.
  source 1..1320: mol_type = genomic DNA
atggagttcttccccgaggtggccaaggtggagtacgccggccccgagagccgcgacgtcctggcgtatagatggtacaacaaggaagaggtagtgatggggaagaaaatgaaggagtggctgaggttctcggtgtgcttttggcatacctttcgcggaaacgggtcggacccctttggcaagcccaccatcacgcaccgcttcgcaggcgacgatggttcggacaccatggagaacgccctccggcgcgttgaggcggcctttgagctctttgtcaagctcggcgtggagttctactcctttcacgacgtcgatgtggcgcctgagggcaagacgctcaaggagacaaacgagaacctggacaagatcacggaccgcatgctcgagctgcaacaggagacgggcgtcaagctgctctggggcactgccaacttgttctctcatccgcgatacatgaacggcgggtcaacaaacccggatcccaaggtctttgtgcgcgccgccgcgcaggtgaaaaaggccatcgacgtgacccacaaactcggtggcgaaggctttgtgttctggggcggtcgggagggttacatgcacattctcaacacggatatggtccgtgaaatgaatcattacgcgaaaatgctcaagatggccatcgcctacaagaaaaagatcggcttcggcgggcagatcctggtcgaacccaagccccgcgagcccatgaagcaccagtatgactacgacgtgcagaccgtcattggctttctcagacagcacggcctggaaaacgaggtcagcctcaacgtggagcccaatcacacgcagctcgccgggcacgagtttgagcacgatgtcgtcctcgccgcgcagctcggcatgctcggcagcgtcgacgccaacacgggctccgagagcctcgggtgggacacggacgagttcatcaccgaccaaacgcgcgccactgtgctttgcaaggccatcattgagatgggtggtttcgttcagggcggtctcaactttgacgccaaggtccgtcgggagagcaccgacccggaggacctctttatcgctcatgtcgcctcgattgacgcgctcgccaagggtctgcgcaacgcttcgcagctcgtttctgacggccgcatgcgcaaaatgctccaggaccggtacgccggctgggatgagggcatcggacaaaagattgagattggggaaacctcgcttgaggacctcgaggcccactgcctgcaggacgacacggaaccagtcaagacgtcggccaagcaggagaaattccttgccgttctcaaccactacatttcctaa

SEQ ID NO: 3 - DNA, 1455 bp, synthetic construct
  source 1..1455: mol_type = other DNA
atgtacatcggcatcgacctcggcacctcgggcgtcaaggtcatcctcctcaacgagcagggcgaggtcgtcgccgcccagaccgagaagctcaccgtctcgcgcccgcacccgctctggtcggagcaggacccggagcagtggtggcaggccaccgaccgcgccatgaaggccctcggcgaccagcactcgctccaggacgtcaaggccctcggcatcgccggccagatgcacggcgccaccctcctcgacgcccagcagcgcgtcctccgcccggccatcctctggaacgacggccgctgcgcccaggagtgcaccctcctcgaggcccgcgtcccgcagtcgcgcgtcatcaccggcaacctcatgatgccgggcttcaccgccccgaagctcctctgggtccagcgccacgagccggagatcttccgccagatcgacaaggtcctcctcccgaaggactacctccgcctccgcatgaccggcgagttcgcctcggacatgtcggacgccgccggcaccatgtggctcgacgtcgccaagcgcgactggtcggacgtcatgctccaggcctgcgacctctcgcgcgaccagatgccggccctctacgagggctcggagatcaccggcgccctcctcccggaggtcgccaaggcctggggcatggccaccgtcccggtcgtcgccggcggcggcgacaacgccgccggcgccgtcggcgtcggcatggtcgacgccaaccaggccatgctctcgctcggcacctcgggcgtctacttcgccgtctcggagggcttcctctcgaagccggagtcggccgtccactcgttctgccacgccctcccgcagcgctggcacctcatgtcggtcatgctctcggccgcctcgtgcctcgactgggccgccaagctcaccggcctctcgaacgtcccggccctcatcgccgccgcccagcaggccgacgagtcggccgagccggtctggttcctcccgtacctctcgggcgagcgcaccccgcacaacaacccgcaggccaagggcgtcttcttcggcctcacccaccagcacggcccgaacgagctcgcccgcgccgtcctcgagggcgtcggctacgccctcgccgacggcatggacgtcgtccacgcctgcggcatcaagccgcagtcggtcaccctcatcggcggcggcgcccgctcggagtactggcgccagatgctcgccgacatctcgggccagcagctcgactaccgcaccggcggcgacgtcggcccggccctcggcgccgcccgcctcgcccagatcgccgccaacccggagaagtcgctcatcgagctcctcccgcagctcccgctcgagcagtcgcacctcccggacgcccagcgctacgccgcctaccagccgcgccgcgagaccttccgccgcctctaccagcagctcctcccgctcatggcctaa

SEQ ID NO: 4 - DNA, 1669 bp, Piromyces sp.
  source 1..1669: mol_type = genomic DNA
gtaaatggctaaggaatatttcccacaaattcaaaagattaagttcgaaggtaaggattctaagaatccattagccttccactactacgatgctgaaaaggaagtcatgggtaagaaaatgaaggattggttacgtttcgccatggcctggtggcacactctttgcgccgaaggtgctgaccaattcggtggaggtacaaagtctttcccatggaacgaaggtactgatgctattgaaattgccaagcaaaaggttgatgctggtttcgaaatcatgcaaaagcttggtattccatactactgtttccacgatgttgatcttgtttccgaaggtaactctattgaagaatacgaatccaaccttaaggctgtcgttgcttacctcaaggaaaagcaaaaggaaaccggtattaagcttctctggagtactgctaacgtcttcggtcacaagcgttacatgaacggtgcctccactaacccagactttgatgttgtcgcccgtgctattgttcaaattaagaacgccatagacgccggtattgaacttggtgctgaaaactacgtcttctggggtggtcgtgaaggttacatgagtctccttaacactgaccaaaagcgtgaaaaggaacacatggccactatgcttaccatggctcgtgactacgctcgttccaagggattcaagggtactttcctcattgaaccaaagccaatggaaccaaccaagcaccaatacgatgttgacactgaaaccgctattggtttccttaaggcccacaacttagacaaggacttcaaggtcaacattgaagttaaccacgctactcttgctggtcacactttcgaacacgaacttgcctgtgctgttgatgctggtatgctcggttccattgatgctaaccgtggtgactaccaaaacggttgggatactgatcaattcccaattgatcaatacgaactcgtccaagcttggatggaaatcatccgtggtggtggtttcgttactggtggtaccaacttcgatgccaagactcgtcgtaactctactgacctcgaagacatcatcattgcccacgtttctggtatggatgctatggctcgtgctcttgaaaacgctgccaagctcctccaagaatctccatacaccaagatgaagaaggaacgttacgcttccttcgacagtggtattggtaaggactttgaagatggtaagctcaccctcgaacaagtttacgaatacggtaagaagaacggtgaaccaaagcaaacttctggtaagcaagaactctacgaagctattgttgccatgtaccaataagttaatcgtagttaaattggtaaaataattgtaaaatcaataaacttgtcaatcctccaatcaagtttaaaagatcctatctctgtactaattaaatatagtacaaaaaaaaatgtataaacaaaaaaaagtctaaaagacggaagaatttaatttagggaaaaaataaaaataataataaacaatagataaatcctttatattaggaaaatgtcccattgtattattttcatttctactaaaaaagaaagtaaataaaacacaagaggaaattttcccttttttttttttttgtaataaattttatgcaaatataaatataaataaaataataaaaaaaaaaaaaaaaaa

SEQ ID NO: 5 - protein, 395 aa, Streptomyces lividans
MNYQPTSEDRFTFGLWTVGWQGLDPFGDATREALDPAESVRRLSQLGAYGVTFHDDELIPFGSSDNERGVAHGAGVAHQAVPAGAGRDRHEGADGDDEPVHAPGCSRDGAFTANDRDVRGTRCARAIRNIDLAVEHVARASTCAWGGREGAESGAAKDVRDALDRMKEAFDLLGEYVTEQGYDLKFAIEPKPNEPRGDILLPTVGHALAFIERLERPELYGVNPEVGHEQMAGLNFPHGIAQALWAGKLFHIDLNGQSGIKYDQDLRFGAGDLRAAFWLVDLLERAGYAGPRHFDFKPPRTENFDAVWPSAAGCMRNYLILKDRAAAFRADPQVQEALAAARLDELARPTAEDGLAALLADRSAYDTFDVDAAAARGMAFEHLDQLAMDHLLGAR

SEQ ID NO: 6 - DNA, 2040 bp, Piromyces sp.
  source 1..2040: mol_type = genomic DNA
attatataaaataactttaaataaaacaatttttatttgtttatttaattattcaaaaaaaattaaagtaaaagaaaaataatacagtagaacaatagtaataatatcaaaatgaagactgttgctggtattgatcttggaactcaaagtatgaaagtcgttatttacgactatgaaaagaaagaaattattgaaagtgctagctgtccaatggaattgatttccgaaagtgacggtacccgtgaacaaaccactgaatggtttgacaagggtcttgaagtttgttttggtaagcttagtgctgataacaaaaagactattgaagctattggtatttctggtcaattacacggttttgttcctcttgatgctaacggtaaggctttatacaacatcaaactttggtgtgatactgctaccgttgaagaatgtaagattatcactgatgctgccggtggtgacaaggctgttattgatgcccttggtaaccttatgctcaccggtttcaccgctccaaagatcctctggctcaagcgcaacaagccagaagctttcgctaacttaaagtacattatgcttccacacgattacttaaactggaagcttactggtgattacgttatggaatacggtgatgcctctggtaccgctctcttcgattctaagaaccgttgctggtctaagaagatttgcgatatcattgacccaaaacttttagatttacttccaaagttaattgaaccaagcgctccagctggtaaggttaatgatgaagccgctaaggcttacggtattccagccggtattccagtttccgctggtggtggtgataacatgatgggtgctgttggtactggtactgttgctgatggtttccttaccatgtctatgggtacttctggtactctttacggttacagtgacaagccaattagtgacccagctaatggtttaagtggtttctgttcttctactggtggatggcttccattactttgtactatgaactgtactgttgccactgaattcgttcgtaacctcttccaaatggatattaaggaacttaatgttgaagctgccaagtctccatgtggtagtgaaggtgttttagttattccattcttcaatggtgaaagaactccaaacttaccaaacggtcgtgctagtattactggtcttacttctgctaacaccagccgtgctaacattgctcgtgctagtttcgaatccgccgttttcgctatgcgtggtggtttagatgctttccgtaagttaggtttccaaccaaaggaaattcgtcttattggtggtggttctaagctgatctctggagacaaattgccgctgatatcatgaaccttccaatcagagttccacttttagaagaagctgctgctcttggtggtgctgttcaagctttatggtgtcttaagaaccaatctggtaagtgtgatattgttgaactttgcaaagaacacattaagattgatgaatctaagaatgctaacccaattgccgaaaatgttgctgtttacgacaaggcttacgatgaatactgcaaggttgtaaatactctttctccattatatgcttaaattgccaatgtaaaaaaaaatataatgccatataattgccttgtcaatacactgttcatgttcatataatcataggacattgaatttacaaggtttatacaattaatatctattatcatattattatacagcatttcattttctaagattagacgaaacaattcttggttccttgcaatatacaaaatttacatgaatttttagaatagtctcgtatttatgcccaataatcaggaaaattacctaatgctggattcttgttaataaaaacaaaataaataaattaaataaacaaataaaaattataagtaaatataaatatataagtaatataaaaaaaaagtaaataaataaataaataaataaaaattttttgcaaatatataaataaataaataaaatataaaaataatttagcaaataaattaaaaaaaaaaaaaaaaaa

SEQ ID NO: 7 - DNA, 1803 bp, Saccharomyces sp.
  source 1..1803: mol_type = genomic DNA
atgttgtgttcagtaattcagagacagacaagagaggtttccaacacaatgtctttagactcatactatcttgggtttgatctttcgacccaacaactgaaatgtctcgccattaaccaggacctaaaaattgtccattcagaaacagtggaatttgaaaaggatcttccgcattatcacacaaagaagggtgtctatatacacggcgacactatcgaatgtcccgtagccatgtggttagaggctctagatctggttctctcgaaatatcgcgaggctaaatttccattgaacaaagttatggccgtctcagggtcctgccagcagcacgggtctgtctactggtcctcccaagccgaatctctgttagagcaattgaataagaaaccggaaaaagatttattgcactacgtgagctctgtagcatttgcaaggcaaaccgcccccaattggcaagaccacagtactgcaaagcaatgtcaagagtttgaagagtgcataggtgggcctgaaaaaatggctcaattaacagggtccagagcccattttagatttactggtcctcaaattctgaaaattgcacaattagaaccagaagcttacgaaaaaacaaagaccatttctttagtgtctaattttttgacttctatcttagtgggccatcttgttgaattagaggaggcagatgcctgtggtatgaacctttatgatatacgtgaaagaaaattcagtgatgagctactacatctaattgatagttcttctaaggataaaactatcagacaaaaattaatgagagcacccatgaaaaatttgatagcgggtaccatctgtaaatattttattgagaagtacggtttcaatacaaactgcaaggtctctcccatgactggggataatttagccactatatgttctttacccctgcggaagaatgacgttctcgtttccctaggaacaagtactacagttcttctggtcaccgataagtatcacccctctccgaactatcatcttttcattcatccaactctgccaaaccattatatgggtatgatttgttattgtaatggttctttggcaagggagaggataagagacgagttaaacaaagaacgggaaaataattatgagaagactaacgattggactctttttaatcaagctgtgctagatgactcagaaagtagtgaaaatgaattaggtgtatattttcctctgggggagatcgttcctagcgtaaaagccataaacaaaagggttatcttcaatccaaaaacgggtatgattgaaagagaggtggccaagttcaaagacaagaggcacgatgccaaaaatattgtagaatcacaggctttaagttgcagggtaagaatatctcccctgctttcggattcaaacgcaagctcacaacagagactgaacgaagatacaatcgtgaagtttgattacgatgaatctccgctgcgggactacctaaataaaaggccagaaaggactttttttgtaggtggggcttctaaaaacgatgctattgtgaagaagtttgctcaagtcattggtgctacaaagggtaattttaggctagaaacaccaaactcatgtgcccttggtggttgttataaggccatgtggtcattgttatatgactctaataaaattgcagttccttttgataaatttctgaatgacaattttccatggcatgtaatggaaagcatatccgatgtggataatgaaaattgggatcgctataattccaagattgtccccttaagcgaactggaaaagactctcatctaa

SEQ ID NO: 8 - protein, 2942 aa, Pichia sp.
TTANACAGTTTTCCAGAATCCAAATTTTCCAACCAACNAAAAACGGACCCAGAAAGTTACAGATTTTTCAGAGCTTCATCTTTTNTANGATTTCACAGCTTCATCAATTTCAGACCATAGCCATAATGACTTTTGTAGAGTTTCCNATCACTATTCCCAACCAGCAGCGTGTGNAAACTGCCATCACCTATAGTGCCTACTTTTCGGTTTTCACCAGTGTGGTTTTTGGCCTAGTTACAAATTCGCTAGAGAATGTTGTGTATGCTTTTGGAGCGCAGACTGCCATCACGTTAGTGTTGACTGCATTCAACTGGCCGTGGTTCACGAGTGCTCCCGGTATCGAATGGCTCCCGGTAGAATTTTAGGATCGTATGGTGACTTGGCGATTTAACTGGGTAGCACAAGGGAATTTTCAGGAAATTTTCTGGTTGGACATTTTGGGCGGCTGAACTTTCATGGTTAAAAGGACTAAGGCCAGATTCTCGGGGGGAGAAAAATTTCTGTTAGTTTGGAATTTTCCGAGCCCCACACATTGCGATGGTAGATTCGGTACGAAACTATATAAACGGTTGGATTCCTAGAAAGGGCCAGATCAGATTGTAGSTAGTATATATAGCATATAGATCCCTGGAGGATACCCACAGACATTACTGCTACTAATTCATACCATACTTGACGTATATCTGCGCATACATATCTACCCCAACTTTCATATAAAATTCCTAGATTTATTGCATCTTCTAATAGAGTCATTTTTCAGATTTTTCAATTTCCATAGAAAGCATACATTTTCATACAGCTTCTATTTGTTAATCGACCTGATAATTTTACTAGCCATATTTCTTTTTTTGATTTTTCACTTAATCGACATATAAATACTCACGTAGTTGACACTCACAATGACCACTACCCCATTTGATGCTCCAGATAAGCTCTTCCTCGGGTTCGATCTTTCGACTCAGCAGTTGAAGATCATCGTCACCGATGAAAACCTCGCTGCTCTCAAAACCTACAATGTCGAGTTCGATAGCATCAACAGCTCTGTCCAGAAGGGTGTCATTGCTATCAACGACGAAATCAGCAAGGGTGCCATTATTTCCCCCGTTTACATGTGGTTGGATGCCCTTGACCATGTTTTTGAAGACATGAAGAAGGACGGATTCCCCTTCAACAAGGTTGTTGGTATTTCCGGTTCTTGTCAACAGCACGGTTCGGTATACTGGTCTAGAACGGCCGAGAAGGTCTTGTCCGAATTGGACGCTGAATCTTCGTTATCGAGCCAGATGAGATCTGCTTTCACCTTCAAGCACGCTCCAAACTGGCAGGATCACTCTACCGGTAAAGAGCTTGAAGAGTTCGAAAGAGTGATTGGTGCTGATGCCTTGGCTGATATCTCTGGTTCCAGAGCCCATTACAGATTCACAGGGCTCCAGATTAGAAAGTTGTCTACCAGATTCAAGCCCGAAAAGTACAACAGAACTGCTCGTATCTCTTTAGTTTCGTCATTTGTTGCCAGTGTGTTGCTTGGTAGAATCACCTCCATTGAAGAAGCCGATGCTTGTGGAATGAACTTGTACGATATCGAAAAGCGCGAGTTCAACGAAGAGCTCTTGGCCATCGCTGCTGGTGTCCACCCTGAGTTGGATGGTGTAGAACAAGACGGTGAAATTTACAGAGCTGGTATCAATGAGTTGAAGAGAAAGTTGGGTCCTGTCAAACCTATAACATACGAAAGCGAAGGTGACATTGCCTCTTACTTTGTCACCAGATACGGCTTCAACCCCGACTGTAAAATCTACTCGTTCACCGGAGACAATTTGGCCACGATTATCTCGTTGCCTTTGGCTCCAAATGATGCTTTGATCTCATTGGGTACTTCTACTACAGTTTTAATTATCACCAAGAACTACGCTCCTTCTTCTCAATACCATTTGTTTAAACATCCAACCATGCCTGACCACTACATGGGCATGATCTGCTACTGTAACGGTTCCTTGGCCAGAGAAAAGGTTAGAGACGAAGTCAACGAAAAGTTCAATGTAGAAGACAAGAAGTCGTGGGACAAGTTCAATGAAATCTTGGACAAATCCACAGACTTCAACAACAAGTTGGGTATTTACTTCCCACTTGGCGAAATTGTCCCTAATGCCGCTGCTCAGATCAAGAGATCGGTGTTGAACAGCAAGAACGAAATTGTAGACGTTGAGTTGGGCGACAAGAACTGGCAACCTGAAGATGATGTTTCTTCAATTGTAGAATCACAGACTTTGTCTTGTAGATTGAGAACTGGTCCAATGTTGAGCAAGAGTGGAGATTCTTCTGCTTCCAGCTCTGCCTCACCTCAACCAGAAGGTGATGGTACAGATTTGCACAAGGTCTACCAAGACTTGGTTAAAAAGTTTGGTGACTTGTTCACTGATGGAAAGAAGCAAACCTTTGAGTCTTTGACCGCCAGACCTAACCGTTGTTACTACGTCGGTGGTGCTTCCAACAACGGCAGCATTATCCSCAAGATGGGTTCCATCTTGGCTCCCGTCAACGGAAACTACAAGGTTGACATTCCTAACGCCTGTGCATTGGGTGGTGCTTACAAGGCCAGTTGGAGTTACGAGTGTGAAGCCAAGAAGGAATGGATCGGATACGATCAGTATATCAACAGATTGTTTGAAGTAAGTGACGAGATGAATCTGTTCGAAGTCAAGGATAAATGGCTCGAATATGCCAACGGGGTTGGAATGTTGGCCAAGATGGAAAGTGAATTGAAACACTAAAATCCATAATAGCTTGTATAGAGGTATAGAAAAAGAGAACGTTATAGAGTAAAGACAATGTAGCATATATGTGCGAATATCACGATAGACGTTATACAGAAGATTACTTTCACATCATTTTGAAAATATCTTGATATGTTCATATTTCATTCGCCTCTAGCATTTTTCAGA

SEQ ID NO: 9 - DNA, 1623 bp, Aspergillus sp.
  source 1..1623: mol_type = genomic DNA
atggctatcggcaatctttacttcattgcggccatcgccgtcgtcggcggtggtctgttcggtttcgatatctcgtcgatgtcggccatcatcgagaccgatgcctatctctgttacttcaaccaggctcctgtcacttacgatgatgatggcaagagggtctgtcagggccccagcgcgagtgtgcagggtggtatcaccgcctccatggctggtggttcctggttgggctcgttgatctcgggtttcatctcggacaggcttggtcgtcgtactgccattcagatcggttccatcatctggtgcattggatccatcattgtctgtgcctcccagaacattcccatgctgatcgtcggtcgtatcatcaacggtctgagtgtgggtatctgctccgctcaggtgccagtgtatatttcggagattgctcctccaaccaagcgtggtcgtgtcgtcggtctgcaacaatgggctattacctggggtatcctgatcatgttctacgtctcctatggatgcagcttcatcaagggtacggcggccttccggattccctggggtctgcagatgatccctgccgtgctattgttcctgggtatgatgctcctgcctgagtcaccccgctggctggcacgcaaggaccgatgggaggagtgccacgctgttttgaccctcgtccacggtcagggagacccgagctctccctttgtgcagcgtgaatatgaagagatcaagagcatgtgcgagtttgagcgccaaaacgcggatgtctcctacctcgagctgttcaagcccaacatgcttaaccgtacccatgtgggtgttttcgttcagatctggtctcagttgactggaatgaacgtcatgatgtactacatcacctacgtctttgccatggccggcttgaaaggtaacaacaacttgatctcctccagtatccagtacgtgatcaacgtgtgcatgactgtgccggctctggtgtggggtgatcagtggggccgtcgcccgaccttcttgatcggttccctcttcatgatgatctggatgtacattaatgctggtctgatggccagctacggtcatcccgcgccgcccggcggtctcaacaacgtggaagccgagtcctgggtcatccacggcgcgcccagcaaggctgtcattgccagtacctacctcttcgtagcctcatacgccatctccttcggccccgccagctgggtgtacccgccggaactcttccctctgcgtgtgcgcggcaaggctaccgccctctgcacttcagccaactgggccttcaacttcgccctcagctattttgtccccccggcatttgtcaacatccagtggaaggtctacatcctcttcggtgtcttctgtactgccatgttcttgcacattttcttcttctttcccgagaccacgggtaagaccctggaagaggtcgaggccatcttcactgatcccaatggtattccgtacatcggtactcccgcctggaagacaaagaacgagtactcgcgcggtgcacacattgaggaggttggctttgaagatgagaagaaggttgctggtgggcagactatccaccaggaggtcacggctactccggataagattgcttga

SEQ ID NO: 10 - DNA, 1644 bp, Candida sp.
  source 1..1644: mol_type = genomic DNA
atgtcacaagattcgcattcttctggtgccgctacaccagtcaatggttccatccttgaaaaggaaaaagaagactctccagttcttcaagttgatgccccacaaaagggtttcaaggactacattgtcatttctatcttctgttttatggttgccttcggtggtttcgtcttcggtttcgacactggtaccatttccggtttcgtgaacatgtctgactttaaagacagattcggtcaacaccacgctgatggtactccttacttgtccgacgttagagttggtttgatgatttctattttcaacgttggttgcgctgtcggtggtattttcctctgcaaggtcgctgatgtctggggtagaagaattggtcttatgttctccatggctgtctacgttgttggtattattattcagatctcttcatccaccaagtggtaccagttcttcattggtcgtcttattgctggtttggctgttggtaccgtttctgtcgtttccccacttttcatctctgaggtttctccaaagcaaattagaggtactttagtgtgctgcttccagttgtgtatcaccttgggtatcttcttgggttactgtactacttacggtactaagacctacactgactctagacagtggagaattcctttgggtttgtgtttcgcttgggctatcttgttggttgtcggtatgttgaacatgccagagtctccaagatacttggttgagaagcacagaattgatgaggccaagagatccattgccagatccaacaagatccctgaggaggacccattcgtctacactgaggttcagcttattcaggccggtattgagagagaagctttggctggtcaggcatcttggaaggagttgatcactggtaagccaaagatcttcagaagagttatcatgggtattatgcttcagtccttgcaacagttgaccggtgacaactacttcttctactacggtactaccattttccaggctgtcggtttgaaggattctttccagacttctatcattttgggtattgtcaactttgcttccaccttcgttggtatctatgtcattgagagattgggtagaagattgtgtcttttgaccggttccgctgctatgttcatctgtttcatcatctactctttgattggtactcagcacttgtacaagcaaggttactccaacgagacctccaacacttacaaggcttctggtaacgctatgatcttcatcacttgtctttacattttcttctttgcttctacctgggctggtggtgtttactgtatcatttccgagtcctacccattgagaattagatccaaggccatgtctattgctaccgctgctaactggttgtggggtttcttgatttccttcttcactccattcatcaccagtgccatccacttctactacggtttcgttttcactggttgtttggctttctctttcttctacgtctacttcttcgtctacgaaaccaagggtctttctttggaggaggttgatgagatgtacgcttccggtgttcttccactcaagtctgccagctgggttccaccaaatcttgagcacatggctcactctgccggttacgctggtgctgacaaggccaccgacgaacaggtttaa

SEQ ID NO: 11 - DNA, 1569 bp, Candida sp.
  source 1..1569: mol_type = genomic DNA
atgggtttggaggacaatagaatggttaagcgtttcgtcaacgttggcgagaagaaggctggctctactgccatggccatcatcgtcggtctttttgctgcttctggtggtgtccttttcggatacgatactggtactatttctggtgtgatgaccatggactacgttcttgctcgttacccttccaacaagcactcttttactgctgatgaatcttctttgattgtttctatcttgtctgttggtactttctttggtgcactttgtgctccattccttaacgacaccctcggtagacgttggtgtcttattctttctgctcttattgtcttcaacattggtgctatcttgcaggtcatctctactgccattccattgctttgtgctggtagagttattgcaggttttggtgtcggtttgatttctgctactattccattgtaccaatctgagactgctccaaagtggatcagaggtgccattgtctcttgttaccagtgggctattaccattggtcttttcttggcctcttgtgtcaacaagggtactgagcacatgactaactctggatcttacagaattccacttgctattcaatgtctttggggtcttatcttgggtatcggtatgatcttcttgccagagactccaagattctggatctccaagggtaaccaggagaaggctgctgagtctttggccagattgagaaagcttccaattgaccacccagactctctcgaggaattaagagacatcactgctgcttacgagttcgagactgtgtacggtaagtcctcttggagccaggtgttctctcacaagaaccaccagttgaagagattgttcactggtgtggctatccaggctttccagcaattgaccggtgttaacttcattttctactacggtactaccttcttcaagagagctggtgttaacggtttcactatctccttggccactaacattgtcaatgtcggttctactattccaggtattcttttgatggaagtcctcggtagaagaaacatgttgatgggtggtgctactggtatgtctctttctcaattgatcgttgccattgttggtgttgctacctcggaaaacaacaagtcttcccagtccgtccttgttgctttctcctgtattttcattgccttcttcgctgccacctggggtccatgtgcttgggttgttgttggtgagttgttcccattgagaaccagagctaagtctgtctccttgtgtactgcttccaactggttgtggaactggggtattgcttacgctactccatacatggtggatgaagacaagggtaacttgggttccaatgtgttcttcatctggggtggtttcaacttggcttgtgttttcttcgcttggtacttcatctacgagaccaagggtctttctttggagcaggtcgacgagttgtacgagcatgtcagcaaggcttggaagtctaagggcttcgttccatctaagcactctttcagagagcaggtggaccagcaaatggactccaaaactgaagctattatgtctgaagaagcttctgtttaa

SEQ ID NO: 12 - DNA, 1662 bp, Pichia sp.
  source 1..1662: mol_type = genomic DNA
atgtctgtagatgaaaatcaattggagaatggacaacttctatcctccgaaaatgaggcatcatcaccttttaaagagtctatcccttctcgctcttccctctacttaatagctcttacagtttcacttttgggagttcaattgacttggtcggttgaacttggttatggtacaccgtatttattctcacttggtcttcgtaaagaatggacttcaattatatggattgccggtcctttgactggaatattaattcagccaattgctggtatattgtccgaccgggttaattcaagaataggtcggcggagaccgttcatgctctgtgctagtttgttaggaacattcagcttattccttatgggctgggcccctgatatttgcctctttatatttagcaatgaggttctaatgaaacgtgttactatcgttttggctacgattagcatttatttgcttgacgtggccgtcaatgtcgtaatggctagcactcgatctttaattgttgattcagtccgttcagatcaacagcatgaagcaaattcctgggctggaagaatgataggtgtaggcaatgtgcttgggtacttactaggctatttacctctatatcgcatcttctcctttctcaatttcacacagttacaggtgttttgcgtacttgcctccatttccttggtactcacagttaccatcacaacaatatttgtgagtgaaaggagattcccaccagttgaacacgagaaatcggttgctggagaaatctttgaattttttacaactatgcgacaaagtattaccgcacttccatttacattaaaaagaatttgttttgttcaattttttgcatactttggatggtttccatttttgttttatattactacctatgtgggtattttatatttacgccatgctcctaaaggccatgaagaagattgggacatggcgactcgtcaagggtcgttcgcattactgctttttgctatcatttctcttgccgcaaatacagcacttccattgttgctcgaggacacggaggatgatgaggaggacgaatcgagtgatgcatctaataatgaatacaacattcaagaaagaaacgatctcggaaatataagaactggtactaatacaccccgtcttggtaatttgagcgaaacaacttctttccgttcggaaaatgagccctcacgacgcaggcttttaccgtctagtagatcaattatgacaacgatatcctccaaggtacaaatcaaaggacttactcttcctattctgtggttgagctcccatgtcctttttggtgtttgtatgttgagcacgatattcttgcaaacatcatggcaagcgcaggcaatggtagctatctgtggactgtcctgggcatgtactctatggattccatattcgctattttcttcagaaatagggaagcttggattacgagaaagcagtggcaaaatgattggtgttcacaatgtatttatatctgccccccaagtgttgagcaccatcattgccaccattgtatttattcaatcggagggcagtcatcgagacatcgccgacaatagtatagcatgggtgttgagaattggaggtatatctgcatttctagccgcgtaccaatgccggcatcttttgcccatcaacttttga